(19) United States
(12) Patent Application Publication
(10) Pub. No.: US 2012/0148541 A1
(43) Pub. Date: Jun. 14, 2012

(54) COMPOSITIONS AND METHODS TO GENERATE PILOSEBACEOUS UNITS

(75) Inventors: Lily LEE, Los Angeles, CA (US); Cheng Ming Chuong, Los Angeles, CA (US); Warren Garner, Los Angeles, CA (US); Ting Xin Jiang, Los Angeles, CA (US)

(73) Assignee: University of Southern California

(21) Appl. No.: 13/283,222

(22) Filed: Oct. 27, 2011

Related U.S. Application Data
(60) Provisional application No. 61/407,865, filed on Oct. 28, 2010.

(51) Int. Cl. A61K 35/36; A61P 17/02; C12Q 1/02; A61P 17/00

(52) U.S. Cl. 424/93.7; 435/29

(57) ABSTRACT

The disclosure describes compositions and methods to generate pilosebaceous units. In one aspect, a biocompatible scaffold and an effective amount of dermal and epidermal precursor cells is described.

### CROSS-REFERENCE TO RELATED APPLICATION

This invention claims priority under 35 U.S.C. §119(e) of U.S. Provisional Application No. 61/407,865, filed Oct. 28, 2010, the contents of which are hereby incorporated by reference in its entirety into the present disclosure.

### STATEMENT OF FEDERAL SUPPORT

This invention was made with government support under Grant Nos. AR 43177; AR 047364; 82019-02 (LFL) and GM050967 (WLG) awarded by the National Institutes of Health. The government has certain rights in the invention.

### BACKGROUND

The ability to reconstitute adult skin with functional skin appendages has long been a major clinical objective for dermatologists and surgeons. Lichti et al., 1993 shows that it is possible to use dissociated hair precursor cells to have de novo formation of hair follicles in vivo. Their protocol is now widely used to mix dissociated multi-potential dermal and epidermal stem cells from newborn mouse skin to form hairs. This assay and its modifications have been used to test the ability of hair bulge stem cells from adult mouse to form hairs (Morris et al., 2004; Tumbar et al., 2004).

Lichti's chamber procedure (Lichti et al., 1993 and Lichti et al., 2008) is an achievement toward the de novo formation of new hair follicles, the procedure is time consuming to perform. Also it requires a specialized chamber to fit the wound shape, and the animal has to carry the cumbersome chamber during the wound healing process of 2-3 weeks. While it is an useful assay for evaluating the efficacy of stem cell candidates to form hairs, it is not practical for a larger scale screening, nor future clinical application. A simplified procedure was developed by injecting much smaller amount of dissociated precursor cells underneath the skin of mice (Zheng et al., 2005). In comparison, this procedure (called patchy assay by the authors) is much easier to perform, and allows for large scale screening. However, most time the patchy assay leads to the formation of subcutaneous hair cysts. Hair filaments pointing inward and the growth of these hair filaments are trapped in the cyst. While these hair follicles cycle, they cannot cycle normally. Thus this procedure is useful for evaluating the efficacy of molecules or candidate cells on hair formation in a short term basis. However, the procedure can not become the basis toward practical clinical applications in the future.

Therefore while useful procedures have been developed and progress are made (Lee et al., 2009), there is still need for developing a simple and high-throughput procedure that can generate a large number of pilosebaceous units with a clinically acceptable appearance. This invention satisfies this need and provides related advantages as well.

### SUMMARY

One of the major objectives of tissue engineering is to reconstitute skin from stem cells. This requires multi-potent skin stem cells and the ability to guide these cells to form a piece of skin with proper architecture and skin appendages. Based on previous progress, Applicants developed a simplified procedure that can be useful for large-scale screening of factors that can modulate the hair formation ability of candidate cells. Newborn mouse cells are used. Dissociated epidermal and dermal cells in high density suspension are allowed to reconstitute in vitro to generate its own matrix, or seeded into a scaffold-like matrix already used clinically (e.g., Integra). These cells self-organize and form a reconstituted skin with proper proportions and topological organization of different components. Large numbers of hair follicles form. The cellular and molecular events are characterized, showing a distinct but parallel morphogenetic process comparing to those occur in embryonic development. The formed hair follicles can cycle and regenerate and the reconstituted skin can heal after injury. The skins are in good condition after one year of transplant. This procedure can also be performed with flexible size and shape of the reconstituted skin, so that clinical applications are possible.

Provided herein is a new procedure that allows multipotential skin precursor cells to form a large number of new hair follicles which are arranged in a physiological plane with a cosmetically acceptable appearance. This procedure can be performed efficiently, reproducibly and on a large scale so as to be appropriate for clinical applications.

Further provided is a method for preparing pilosebaceous units in a physiological plane, comprising, or alternatively consisting essentially of, or yet further consisting of, admixing a number of skin precursor cells and a suitable medium, wherein the concentration of skin precursor cells present in the medium is from about 0.5 million cells per 100 μl medium to about 40 million cells in 300 μl medium. In one aspect, the concentration of cells in the medium is from about 2 million cells per 150 μl medium to about 20 million cells in 200 μl medium. In one aspect, the suitable medium is a serum-free medium.

In another aspect, the method further comprising, or alternatively consisting essentially of, or yet further consisting of, culturing the cells in the medium for an effective amount of time to allow the cells to settle and excess liquid to be removed.

In a yet further aspect, the effective amount of time is from about 30 minutes to about 4 hours at a temperature that supports cell stability. The temperature in a range from about 34° C. to about 40° C., or alternatively in a range from about 36° C. to about 38° C. In a yet further aspect, the temperature is about 37° C.+/−0.5, or 0.4, or 0.3, or 2, or 0.1° C.

The disclosure also provides a method wherein the skin precursor cells comprises, or alternatively consisting essentially of, or yet further consists of, epidermal and dermal precursor cells. For example, the epidermal and dermal precursor cells are isolated or purified cells from neonatal or aged mammals.

In a further aspect, the ratio of epidermal to dermal precursor cells is from about 1:3 to about 1:15, or alternatively, from about 1:5 to about 1:10, and increments within these ranges.

The method can further comprise, or alternatively consists essentially of, or yet further consist of, admixing an effective amount of an agent inhibiting Bone Morphogenic Protein (BMP) signaling. The agent can be selected from the group consisting of dorsomorphin, noggin, chordin, gremlin, sclerostin, follistatin and combinations thereof.

The method can further comprise, or alternatively consists essentially of, or yet further consist of, admixing an effective amount of an agent promoting cell differentiation or growth. The agent can selected from the group consisting of Platelet Derived Growth Factor (PDGF), Vascular Endothelial Growth Factor (VEGF), Epithelial Growth Factor (EGF), TGF-, Fibroblast Growth Factor (FGF), insulin, transferrin, retinoid, and combinations thereof.

The method can further comprise, or alternatively consists essentially of, or yet further consist of, admixing an effective amount of minoxidil, finasteride, or an agent enhancing cell growth.

In a yet further aspect, the method can further comprise, or alternatively consist essentially of, or yet further consist of, seeding the cells in the medium onto a biocompatible scaffold. In one aspect, the biocompatible scaffold is dried or lyophilized prior to admixing with the cells in serum-free medium. In another aspect, the cells are seeded by passively contacting the cells with the scaffold at a temperature range from about 25° C. to about 40° C. for about 30 minutes to about 2 hours.

In a yet further aspect, the method further comprises, or alternatively consists essentially of, or yet further consists of, implanted into the cells in the suitable medium onto or into the dermis of the non-human animal.

In another aspect, the method further comprises, or alternatively consists essentially of, or yet further consists of, admixing an agent to be screened, and monitoring the growth of hair in vitro or in vivo.

The method can be further modified by implanting the cells and suitable medium into the dermal layer of the mammal under a condition that favors implantation of the composition into the dermis of the mammal. For example, the conditions that favor implantation of the composition into the dermis of the mammal comprises or alternatively consists essentially of, or yet further consists of, applying suitable pressure to maintain contact between the composition and the muscle or subcutaneous fat of the mammal for at least 3 days. In one aspect, the dermal layer of the mammal was pretreated with an effective amount of an agent inhibiting the Bone Morphogenic Protein (BMP) signaling. The agent is one or more of dorsomorphin, noggin, chordin, gremlin, sclerostin, or follistatin.

### BRIEF DESCRIPTION OF THE FIGURES

FIG. 1, panels A through L illustrate a step by step pictorial of the protocol. (A) Mice are cleaned and prepared for surgery under anesthesia. (B and C) The approximate area of skin to be grafted for hair bearing is cut out in a full thickness layer, note musculature. (D) An example of scaffold that has been seeded with cells using a pipet and allowed to dry briefly. Scaffold is sitting on a protective silicone membrane. (E) Placement of scaffold and cells over recipient bed. (F and G) Simple interrupted sutures to secure graft in place. (H and I) Antimicrobial ointment and gauze used to dress the wound. (J, K and L) Securing of dressing with a tight elastic wrapping allows for better adherence to wound. The mice have no restrictions during the post operative period. Dressings are removed 7-12 days later. The silicone protective layer is easily peeled off once the wound has re-epithelialized.

FIG. 2, panels A through E show generation of planar arranged hairs. (A) Macroscopic evidence of hair growth as soon as dressings are removed on day 11. (B and C) Full growth of hair over grafted region by day 21. (D) Close up view of the hairs. (E) The number of experiments and outcome. +++, the transplanted area is all covered by hairs. ++, >75% of the transplanted area is covered by hairs. +, >50% of the transplanted area is covered by hairs.

FIG. 3 shows molecular characterization of the reconstituted skin. Sections of the reconstituted skin and immuno-statining of molecular markers. Involcurin are in the epidermis. NCAM is mainly in the dermal papilla. Oil 0 Red is shown in sebaceous glands and subcutatenous adipose tissue. AE 13 shows inner root sheath. K14 are in the epidermis and outer root sheath. Versican is in the dermal papilla.

FIG. 4 illustrates cellular and molecular events during the process of hair reconstitution. H&E staining reveals that cells start at the base of the scaffold near the wound bed and migrate to the surface as the cells differentiate and organize themselves into pilosebaceous units within normal skin. K14: There is evidence of basal keratinocytes scattered throughout the matrix initially. They then organize themselves into a basal epidermal layer. NCAM: Positive cells organize themselves over the course of time to the subepidermal layer. Involucrin: Positive cells organize themselves into the basal epidermal layer reconstituting normal epidermis and hair shaft. Versican: Positive cells begin in the same layer as all other cells and by day 8 have homed to the dermal papilla. Note that hair follicle orientation is then readjusted toward the epidermal interface.

FIG. 5, panels A and B show that reconstituted hairs can regenerate. (A.) Physiological hair cycling. Skin is 6 months after planar hair transplantation. At time 0, hairs were clipped with a trimmer. At 2, 3, 4 weeks later, hair were again clipped and photo taken. The pigmented area represents anagen, while the pink area represents telogen. Note pigmented regions in 2 week picture have become pink in 3 week photo. Similarly pigmented regions change in 4 week photo, implying hair cycling changes. (B) Hair regeneration after plucking. Mouse is one year after planar hair transplantation. The reconstituted skin was stripped with warm wax which is similar to plucking over a large area. After hair plucking, region is pinkish (time 0). At 2 week, region show pigmentation, implying follicles below have entered anagen. At 3 and 4 weeks after plucking, hair continues to grow.

FIG. 6, panels A and B show that reconstituted skin is able to respond to full thickness wound and heal. (A) Approximately 3 mm full thickness wound were produced (arrow). 3 days after wounding the opened wound can be seen. 5 days after wounding, the wounds became smaller. 10 days after wounding, the wound is closed. Note, the hairs to the left of the wound seem to grow robustly. (B) Diagram showing the rate of wound closure. pwd, days post wounding.

FIG. 7 shows long term survival of reconstituted skin. After planar hair transplantation, the wound area is recovered by regenerative skin. The skin was traced 4, 9, and 12 months after the procedure. Left column: gross view. The 9 month specimen has two small and one big graft. Middle column, skin sections including the wound margin (marked by arrow). In these specimens, hair follicles are in telogen stage. The right column shows reconstituted skin with anagen hair follicles. The skin shows normal architecture with subcutaneous adipose tissue, but not muscle. Duration after the graft and procedure used are indicated.

FIG. 8, panels A through D show the future potentials of reconstituted skin. (A). Possibility for high throughput screening of genes important for becoming multipotential skin cells. Lentivirus was used to transduced cells and hairs can still form. GFP indicate cells were transduced. (B through D). Shaping advantage of this method. Using a reasonably stiff matrix such as Integra to hold multipotential cells, cells can be grafted and specific shapes and sizes for cosmetic applicability. The graft can also be placed virtually any portion of the body.

FIG. 9 is a schematic drawing of the hair reconstitution process in this procedure. Light gray, epidermal cells. Dark gray, dermal cells. Cells are mixed randomly in the three dimensional matrix. Epidermal cells begin to sort themselves out and coalesce to form a layer first near the bottom of the matrix. They then “rise” from the base to the level of the air surface level. Some transient micro epidermal “cysts” can be observed. Dermal cells also start to form condensations adjacent to the “rising” epidermis. The epidermis eventually flattened out at the surface. Hair germs appear periodically. They progress to form hair pegs, and then onto form hair follicles. The morphogenetic process occurs between about day 5 to day 12 after grafting.

### DETAILED DESCRIPTION

Throughout this disclosure, various publications, patents and published patent specifications are referenced by an identifying citation. Also within this disclosure are Arabic numerals referring to referenced citations, the full bibliographic details of which are provided immediately preceding the claims. The disclosures of these publications, patents, and published patent specifications are hereby incorporated by reference in their entirety into the present disclosure to more fully describe the state of the art to which this invention pertains.

### DEFINITIONS

All numerical designations, e.g., pH, temperature, time, concentration, and molecular weight, including ranges, are approximations which are varied (+) or (−) by increments of 1.0 or 0.1, as appropriate. It is to be understood, although not always explicitly stated that all numerical designations are preceded by the term “about”. It also is to be understood, although not always explicitly stated, that the reagents described herein are merely exemplary and that equivalents of such are known in the art.

As used in the specification and claims, the singular form “a”, “an” and “the” include plural references unless the context clearly dictates otherwise. For example, the term “a cell” includes a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this invention or process steps to produce a composition or achieve an intended result. Embodiments defined by each of these transition terms are within the scope of this invention.

The term “isolated” or “purified” means separated from constituents, cellular and otherwise, in which the cell, tissue, polynucleotide, peptide, polypeptide, protein, antibody or fragment(s) thereof, which are normally associated in nature. For example, an isolated polynucleotide is separated from the 3′ and 5′ contiguous nucleotides with which it is normally associated in its native or natural environment, e.g., on the chromosome. As is apparent to those of skill in the art, a non-naturally occurring polynucleotide, peptide, polypeptide, protein, antibody or fragment(s) thereof, does not require “isolation” to distinguish it from its naturally occurring counterpart. An isolated cell is a cell that is separated form tissue or cells of dissimilar phenotype or genotype.

As used herein, the term “Pilosebaceous Unit” refers to the structure present on the surface of mammalian skin consisting of hair follicle, hair shaft and sebaceous gland. Pilosebaceous units are considered as an important pathway for percutaneous absorption of topically applied drugs and delivery systems. Pilosebaceous units are also the structural units for hair growth. For structural and functional descriptions of pilosebaceous units, see Singh et al. (2000) Indian J. Pharmacol. 32:269-281.

As used herein, the term “physiological plane” or “topological plane” refers to the physiological orientation of hair growth, in which the hairs grow towards the outside of the skin of the subject rather than on the underside resulting in formation of cysts.

As used herein, “stem cell” defines a cell with the ability to divide for indefinite periods in culture and give rise to specialized cells. At this time and for convenience, stem cells are categorized as somatic (adult) or embryonic. A somatic stem cell is an undifferentiated cell found in a differentiated tissue that can renew itself (clonal) and (with certain limitations) differentiate to yield all the specialized cell types of the tissue from which it originated. An embryonic stem cell is a primitive (undifferentiated) cell from the embryo that has the potential to become a wide variety of specialized cell types. An embryonic stem cell line is one that has been cultured under in vitro conditions that allow proliferation without differentiation for months to years. Non-limiting examples of embryonic stem cells are the HES2 (also known as ES02) cell line available from ESI, Singapore and the H1 (also know as WA01) cell line available from WiCells, Madison, Wis. Pluripotent embryonic stem cells can be distinguished from other types of cells by the use of markers including, but not limited to, Oct-4, alkaline phosphatase, CD30, TDGF-1, GCTM-2, Genesis, Germ cell nuclear factor, SSEA1, SSEA3, and SSEA4.

A clone is a line of cells that is genetically identical to the originating cell; in this case, a stem cell. “Clonal proliferation” refers to the growth of a population of cells by the continuous division of single cells into two identical daughter cells and/or population of identical cells.

A “precursor” or “progenitor cell” intends to mean cells that have a capacity to differentiate into a specific type of cell. A progenitor cell may be a stem cell. A progenitor cell may also be more specific than a stem cell. A progenitor cell may be unipotent or multipotent. Compared to adult stem cells, a progenitor cell may be in a farther stage of cell differentiation. Progenitor cells are often found in adult organisms, they act as a repair system for the body. Examples of progenitor cells include, but are not limited to, satellite cells found in muscles, intermediate progenitor cells formed in the subventricular zone, bone marrow stromal cells, periosteum progenitor cells, pancreatic progenitor cells and angioblasts or endothelial progenitor cells. Examples of progenitor cells may also include, but are not limited to, epidermal and dermal cells from neonatal organisms.

As used herein, a “pluripotent cell” defines a less differentiated cell that can give rise to at least two distinct (genotypically and/or phenotypically) further differentiated progeny cells. In another aspect, a “pluripotent cell” includes a Induced Pluripotent Stem Cell (iPSC) which is an artificially derived stem cell from a non-pluripotent cell, typically an adult somatic cell, produced by inducing expression of one or more stem cell specific genes Such stem cell specific genes include, but are not limited to, the family of octamer transcription factors, i.e. Oct-3/4; the family of Sox genes, i.e. Sox1, Sox2, Sox3, Sox 15 and Sox 18; the family of Klf genes, i.e. Klf1, Klf2, Klf4 and Klf5; the family of Myc genes, i.e. c-myc and L-myc; the family of Nanog genes, i.e. OCT4, NANOG and REX1; or LIN28. Examples of iPSCs are described in Takahashi et al. Cell advance online publication 20 Nov. 2007 131(5):861-72, 2007; Takahashi & Yamanaka Cell 126:663-76, 2006; Okita et al. Nature 448:260-262, 2007; Yu et al. Science advance online publication 20 Nov. 2007 318(5858):1917-20, 2007; and Nakagawa et al. Nat. Biotechnol. Advance online publication 30 Nov. 2007 26(1):101-6, 2008.

A “multi-lineage stem cell” or “multipotent stem cell” refers to a stem cell that reproduces itself and at least two further differentiated progeny cells from distinct developmental lineages. The lineages can be from the same germ layer (i.e. mesoderm, ectoderm or endoderm), or from different germ layers. An example of two progeny cells with distinct developmental lineages from differentiation of a multilineage stem cell is a myogenic cell and an adipogenic cell (both are of mesodermal origin, yet give rise to different tissues). Another example is a neurogenic cell (of ectodermal origin) and adipogenic cell (of mesodermal origin).

A skin precursor cell intends a pluripotent stem or progenitor cell with the ability to differentiate into at least one of epidermal, dermal and hair tissue types. A multipotent skin precursor cell is identified by one or more markers such as sca-1, fibronectin, p63, S100A6, keratin 19 (K19), SOX2 or β₁ integrin.

An “epidermal precursor cell” as used herein intends cells having the potential to differentiate into epidermal cells. Typically, these cells are identified by the marker β₁ integrin.

A “dermal precursor cell” as used herein intends cells having the potential to differentiate into dermal cells. Typically these cells are identified by one or more of the markers p63, S100A6 or β₁ integrin.

The term “propagate” means to grow or alter the phenotype of a cell or population of cells. The term “growing” refers to the proliferation of cells in the presence of supporting media, nutrients, growth factors, support cells, or any chemical or biological compound necessary for obtaining the desired number of cells or cell type. In one embodiment, the growing of cells results in the regeneration of tissue.

The term “culturing” refers to the in vitro propagation of cells or organisms on or in media of various kinds. It is understood that the descendants of a cell grown in culture may not be completely identical (i.e., morphologically, genetically, or phenotypically) to the parent cell. By “expanded” is meant any proliferation or division of cells.

As used herein, the “lineage” of a cell defines the heredity of the cell, i.e. its predecessors and progeny. The lineage of a cell places the cell within a hereditary scheme of development and differentiation.

A derivative of a cell or population of cells is a daughter cell of the isolated cell or population of cells. Derivatives include the expanded clonal cells or differentiated cells cultured and propagated from the isolated stem cell or population of stem cells. Derivatives also include already derived stem cells or population of stem cells.

“Differentiation” describes the process whereby an unspecialized cell acquires the features of a specialized cell such as a heart, liver, or muscle cell. “Directed differentiation” refers to the manipulation of stem cell culture conditions to induce differentiation into a particular cell type. “Dedifferentiated” defines a cell that reverts to a less committed position within the lineage of a cell. As used herein, the term “differentiates or differentiated” defines a cell that takes on a more committed (“differentiated”) position within the lineage of a cell. As used herein, “a cell that differentiates into a mesodermal (or ectodermal or endodermal) lineage” defines a cell that becomes committed to a specific mesodermal, ectodermal or endodermal lineage, respectively. Examples of cells that differentiate into a mesodermal lineage or give rise to specific mesodermal cells include, but are not limited to, cells that are adipogenic, leiomyogenic, chondrogenic, cardiogenic, dermatogenic, hematopoetic, hemangiogenic, myogenic, nephrogenic, urogenitogenic, osteogenic, pericardiogenic, or stromal.

Examples of cells that differentiate into ectodermal lineage include, but are not limited to epidermal cells, neurogenic cells, and neurogliagenic cells.

Examples of cells that differentiate into endodermal lineage include, but are not limited to pleurogenic cells, and hepatogenic cells, cells that give rise to the lining of the intestine, and cells that give rise to pancreogenic and splanchogenic cells.

The term “neonatal” intends a newborn mammal. In one aspect, a neonatal human is a human infant during the first month after birth. An “aged” mammal refers to an grown up or adult mammal.

“Bone Morphogenic Proteins” (BMP) are a group of multifunctional growth factors and cytokines with effects in various tissues. For example, BMPs are known to induce the formation of bone and/or cartilage. Examples of BMP may include, but are not limited to BMP1, BMP2, BMP3, BMP4, BMP5, BMP6, BMP7, BMP8a, BMP8b, BMP10 and BMP15.

“BMP signaling” or “BMP signaling pathway” refers to the enzyme linked receptor protein signaling transduction pathway involving proteins that directly or indirectly regulate (activate or inhibit) downstream protein activity or gene expression. Examples of molecules involved in the BMP signaling pathways may be found in the public Gene Ontology (GO) database, under GO ID: GO:0030509, accessible at the web page (amigo.geneontology.org/cgi-bin/amigo/term-details.cgi?term=GO:0030509&session_id=5573amigo1226631957), last accessed on Nov. 17, 2008. Without limitation, examples of proteins in the BMP signaling pathway include Activin receptor type-1 (ACVR1, UniProt: Q04771), Activin receptor type-2A (ACVR2A, UniProt: P27037), Activin receptor type-2B (ACVR2B, UniProt: Q13705), BMP1 (UniProt: P13497), BMP2 (UniProt: P12643), BMP3 (UniProt: P12645), BMP4 (UniProt: P12644), BMP5 (UniProt: P22003), BMP6 (UniProt: P22004), BMP7 (UniProt: P18075), BMP8a (UniProt: Q7Z5Y6), BMP8b (UniProt: P34820), BMP10 (UniProt: O95393), BMP15 (UniProt: O95972), Bone morphogenetic protein receptor type-1A (BMPR1A, UniProt: P36894), Bone morphogenetic protein receptor type-1B (BMPR1B, UniProt: O00238), Bone morphogenetic protein receptor type-2 (BMPR2, UniProt: Q13873), Chordin-like protein (CHRDL1, UniProt: Q9BU40), Follistatin-related protein 1 (FSTL1, UniProt: Q12841), Growth/differentiation factor 2 (GDF2, UniProt: Q9UK05), Growth/differentiation factor 6 (GDF6, UniProt: Q6KF10), Growth/differentiation factor 7 (GDF7, UniProt: Q7Z4P5), Gremlin-2 (GREM2, UniProt: Q9H772), RGM domain family member B (RGMB, UniProt: Q6NW40), Ski oncogene (SKI, UniProt: P12755), Mothers against decapentaplegic homolog 4 (SMAD4, UniProt: Q13485), Mothers against decapentaplegic homolog 5 (SMAD5, UniProt: Q99717), Mothers against decapentaplegic homolog 6 (SMAD6, UniProt: 043541), Mothers against decapentaplegic homolog 7 (SMAD7, UniProt: O15105), Mothers against decapentaplegic homolog 9 (SMAD9, UniProt: O15198), E3 ubiquitin-protein ligase SMRF2 (SMURF2, UniProt: Q9HAU4), TGF-beta receptor type III (TGFBR3, UniProt: Q03167), Ubiquitin-conjugating enzyme E2 D1 (UBE2D1, UniProt: P51668), Ubiquitin-conjugating enzyme E2 D3 (UBE2D3, UniProt: P61077) and Zinc finger FYVE domain-containing protein 16 (ZFYVE16, UniProt: Q7Z3T8). Proteins that positively or negatively regulate the BMP signaling, for purpose of this invention, are also considered within the meaning of the BMP signaling. Proteins that positively regulate BMP signaling include, but are not limited to, Serine/threonine-protein kinase receptor R3 (ACVRL1, UniProt: P37023) and Endoglin (ENG, UniProt: P17813). Proteins that negatively regulate BMP signaling include, but are not limited to, Chordin (CHRD, UniProt: Q9H2×0), E3 ubiquitin-protein ligase SMURF1 (SMURF1, UniProt: Q9HCE7), Sclerostin (SOST, UniProt: Q9BQB4) and Brorin (VWC2, UniProt: Q2TAL6). Examples of proteins in the BMP signaling pathway may also include Proprotein convertase subtilisin/kexin type 6 (PCSK6, UniProt: P29122) that regulates BMP signaling.

Small molecules, polynucleotides, polypeptides that enhance or inhibit BMP signaling exist or can be made with procedures known by those skilled in the art. Yanagita (2009) BioFactors 35(2):113-199 is a review article discussing BMP regulators (incorporated by reference). For example, dorsomorphin is a potent small molecule BMP antagonist (Hao et al. (2008) PLoS ONE, 3(8):e2904, Yu et al. (2008) Nat Chem. Biol. 4(1):33-41). Dorsomorphin is currently commercially available from several vendors. Dorsomorphin was reported to selectively inhibit the BMP receptors, type I: ALK2, ALK3 and ALK6 and thus “blocks BMP-mediated SMAD1/5/8 phosphorylation”. Dorsomorphin has preferential specificity toward inhibiting BMP versus TGF-beta and activin signaling. In published reports, dorsomorphin is characterized by low toxicity. It can be delivered into skin to lower macro-environmental BMP signaling and create favorable conditions for hair growth to occur. A unique property of dorsomorphin is that it is a small molecule and is soluble in DMSO. DMSO is known to significantly facilitate trans-dermal delivery of small molecule drugs. This enhancing effect of DMSO on skin penetration can be used in non-invasive method of pharmacological modulation of dermal macro-environment. Treatment procedure thus consists of simply applying liquid form of dorsomorphin in DMSO onto the surface of intact skin. Dorsomorphin in DMSO can be made in form of cream that can be simply rubbed onto intact skin. Small molecule agonist and antagonists for other signaling pathways also exist and can be used to augment or inhibit BMP signaling. Interaction of these small molecules with pathways including, but not limited to, WNT, SHH and FGF will also have direct or indirect impact on BMP signaling thus serve as effective modulator of hair growth via methods disclosed in this invention.

Other types of BMP agonists or antagonists also exist. Yanagita (2009) BioFactors 35(2):113-199 is a review article discussing BMP regulators (incorporated by reference). Non-limiting examples include such as noggin, chordin, gremlin, sclerostin and follistatin. Representative sequences for these proteins include UniProt: Q13253 for noggin, UniProt: Q9H2X0 for chordin, UniProt: 060565 for gremlin, UniProt: Q9BQB4 for sclerostin, and UniProt: P19883 for follistatin. Noggin (UniProt: Q13253), for example, can be produced using methods described in, e.g. McMahon et al. (1998) Genes & Development 12:1438-52.

In some aspects, an agent that can augment or inhibit BMP signaling is a small molecule agonist or antagonist to a BMP agonist or antagonist. In one aspect, the small molecule is a noggin agonist. In another aspect, the small molecule is a noggin antagonist.

Examples of agents that can augment or inhibit BMP signaling also include, but are not limited to, polynucleotides that encode BMP proteins, encode polypeptides augmenting or inhibiting BMP signaling, or augmenting or inhibit expression of BMP proteins, or polypeptides augmenting or inhibiting BMP signaling. In some embodiments, the agent is small interference RNA (siRNA) or double strand RNA (dsRNA) that inhibits expression of proteins that augment or inhibit BMP signaling.

Examples of agents that can augment or inhibit BMP signaling may also include, but are not limited to, an isolated or recombinant BMP protein, or isolated or recombinant polypeptide enhancing or inhibiting BMP signaling. In some aspect, the agent further comprises a pharmaceutically acceptable carrier. In another aspect, the compositions contain carriers that modulate (controlled release) the release of the active agent when administered to a subject in need thereof.

Examples of polypeptide agents that augment or inhibit BMP signaling may also include, but are not limited to, antibodies or modified antibodies including, but not limited to, blocking fragments of antibodies, that activate, stabilize or inhibit proteins in the BMP signaling pathway or proteins modulating the BMP signaling pathway, thereby augmenting or inhibiting BMP signaling.

As used herein, the term “modulate” refers to an act by an agent to regulate, to control or to change certain characteristics of the formation of pilosebaceous units. Examples of the agent may include, but are not limited to, proteins or polypeptides, DNA, RNA, siRNA, dsRNA or other polynucleotides, small molecules. The agent may also mean a temperature change, physical movement or stimulus or any other therapeutic or clinical means that alter the formation of pilosebaceous units. Without limitation, the object may mean a biochemical molecule or pathway, a biochemical activity, a medical condition or any other chemical, biochemical, physical or medical aspect of a subject. In one aspect, the term “modulate” means to enhance the formation of pilosebaceuous units in a plane. In another aspect, the term “modulate” means to inhibit the formation of pilosebaceous units on a plane.

The terms “inhibit” or “antagonize” intend mean an decrease of amount or formation of pilosebaceous units on a plane.

An “agonist”, as used herein, refers to a drug or other chemical that can bind a receptor on a cell to produce a physiologic reaction typical of a naturally occurring substance. The efficacy of an agonist may be positive, causing an increase in the receptor's activity or negative causing a decrease in the receptor's activity.

An “antagonist” refers to a type of receptor ligand or drug that does not provoke a biological response itself upon binding to the receptor, but blocks or dampens agonist-mediated responses. In pharmacology, antagonists have affinity but no efficacy for their cognate receptors and binding will disrupt the interaction and inhibit the function of an agonist or inverse agonist at receptors. Antagonists mediate their effects by binding to the active site or to allosteric sites on receptors or they may interact at unique binding sites not normally involved in the biological regulation of the receptor's activity. Antagonist activity may be reversible or irreversible depending on the longevity of the antagonist-receptor complex which in turn depends on the nature of antagonist receptor binding. The majority of drug antagonists achieve their potency by competing with endogenous ligands or substrates at structurally defined binding sites on receptors.

The term “hair growth” intends to include, but not limited to, the formation of new hair or growth of existing hair.

“Spironolactone” (IUPAC name: 7α-Acetylthio-3-oxo-17α-pregn-4-ene-21,17-carbolactone is marketed under the trade names Aldactone, Novo-Spiroton, Aldactazide, Spiractin, Spirotone, Verospiron or Berlactone) is a diuretic and is used as an antiandrogen. It is also used for treating hair loss in women and can be used as a topical medication for treatment of male baldness.

“Minoxidil” (trade names Rogaine and Regaine; IUPAC name: 6-piperidin-1-ylpyrimidine-2,4-diamine 3-oxide) a commercially available topical formulation that inhibits hair loss, is a vasodilator medication that is available over the counter for treatment of androgenic alopecia, among other baldness treatments.

“Finasteride” (IUPAC name N-(1,1-dimethylethyl)-3-oxo-(5α,17β)-4-azaandrost-1-ene-17-carboxamide) is a synthetic antiandrogen that acts by inhibiting type II 5-alpha reductase, the enzyme that converts testosterone to dihydrotestosterone (DHT). It is used to treat prostate cancer and is registered in many countries to treat adrogenetic alopecia or male pattern baldness. “Propecia,” a medicament containing finasteride as an active ingredient, is commercially available from Merck & Co., Inc.

“Administration”, as used herein, refers to the delivery of a medication or matrix composition to a mammal or subject to be treated and/or in need of such treatment. Non-limiting examples include oral dosing, intracutaneous injection, direct application to target area proximal areas on the skin, or applied on a patch. Various physical and/or mechanical technologies are available to permit the sustained or immediate topical or transdermal administration of macromolecules (such as, peptides). Such technologies include iontophoresis (see for example Kalia et al. (2004) Adv. Drug Del. Rev. 56:619-58) sonophoresis, needle-less injection, and/or microstructured arrays (sometimes called microneedles; one particular example is the Microstructured Transdermal System (MTS) commercially available from 3M) (see, e.g., Alain et al. (2002) J. Control. Release 81:113-119; Santi et al. (1997) Pharm. Res. 14(1):63-66; Sebastien et al. (1998) J. Pharm. Sci. 87(8):922-925). Methods of making and using arrays of solid microneedles that can be inserted into the skin for transdermal delivery of peptides (such as cyclic CRF antagonists) are provided in Martanto et al. (2004) Pharm. Res. 21:947-52, and Martano et al. (2005) Am. Inst. Chem. Eng. 51:1599-607. In some examples, the delivery system includes a combination of systems, such as microneedles made of biocompatible and biodegradable polymers (Park et al. (2005) J. Control. Release 104:51-66). In one aspect, administration is topical administration as defined herein.

“Topical administration” refers to delivery of a composition or medication by application to the skin. Non-limiting examples of topical administration include any methods described under the definition of “administration” pertaining to delivery of a medication to the skin.

A “composition” is intended to mean a combination of active agent, cell or population of cells and another compound or composition, inert (for example, a detectable agent or label or biocompatible scaffold) or active, such as a growth and/or differentiation factor.

A “pharmaceutical composition” is intended to include the combination of an active agent with a carrier, inert or active such as a biocompatible scaffold, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers, and adjuvants see Martin, Remington's Pharm. Sci., 15th Ed. (Mack Publ. Co., Easton (1975)). The term includes carriers that facilitate controlled release of the active agent as well as immediate release.

For topical use, the pharmaceutically acceptable carrier is suitable for manufacture of creams, ointments, jellies, gels, solutions, suspensions, etc. Such carriers are conventional in the art, e.g., for topical administration with polyethylene glycol (PEG). These formulations may optionally comprise additional pharmaceutically acceptable ingredients such as diluents, stabilizers, and/or adjuvants.

The pharmaceutically acceptable carrier facilitate immediate or controlled release of the active ingredient.

“An effective amount” refers to the amount of cells or a biological or chemical agent sufficient to induce a desired biological and/or therapeutic result. That result can be alleviation of the signs, symptoms, or causes of a disease, or any other desired alteration of a biological system. In one aspect, the result of an effective amount of skin precursor cells can include generation of pilosebaceous unites in a physiological plane. In another aspect, the result of an effective amount of an agent inhibiting the BMP signaling can be inhibition of BMP signaling. In yet another aspect, an effective amount of an a gene promoting cell differentiation can be promotion of cell differentiation. The effective amount will vary depending upon the specific cell type or agents used, the desired size or stage of the generated pilosebaceous units, the manner of administration and the like, all of which can be determined readily by one of ordinary skill in the art.

An “epithelial sheet” refers to a biological dressing composed of epidermal keratinocytes and formed in culture as three-dimensional sheet have which has been used for wound healing as skin grafts (See, e.g., U.S. Pat. Nos. 5,292,655; 5,686,307; 5,834,312; 5,912,175; 6,162,643; and 7,037,721).

A “subject” of diagnosis or treatment is a cell or a mammal, including a human. Non-human animals subject to diagnosis or treatment include, for example, murine, such as rats, mice, canine, such as dogs, leporids, such as rabbits, bovine, simian, ovine, livestock, sport animals, and pets.

### Embodiments

Thus, in one aspect this invention provides a composition and method useful in one aspect to generate pilosebaceous units in a physiological plane comprising, or alternatively consisting essentially of, or yet further consisting of, a suitable medium to grow and/or support the cells and an effective amount of skin precursor cells. In one aspect, the method comprises, or alternatively consists essentially of, or yet further consists of, admixing a number of skin precursor cells and a suitable medium, wherein the concentration of skin precursor cells present in the medium is from about 0.5 million cells per 100 μl medium to about 40 million cells in 300 μl medium. In one aspect, the concentration of cells in the medium is from about 2 million cells per 150 μl medium to about 20 million cells in 200 μl medium. Compositions prepared by this method are further provided as well as the use to generate pilosabeceous units.

In another aspect, the method further comprising, or alternatively consisting essentially of, or yet further consisting of, culturing the cells in the medium for an effective amount of time to allow the cells to settle and excess liquid to be removed.

In a yet further aspect, the effective amount of time is from about 30 minutes to about 4 hours at a temperature that supports cell stability. The temperature in a range from about 34° C. to about 40° C., or alternatively in a range from about 36° C. to about 38° C. In a yet further aspect, the temperature is about 37° C. +/−0.5, or 0.4, or 0.3, or 2, or 0.1° C.

The disclosure also provides a method wherein the skin precursor cells comprises, or alternatively consisting essentially of, or yet further consists of, epidermal and dermal precursor cells. For example, the epidermal and dermal precursor cells are isolated or purified cells from neonatal or aged mammals.

In some embodiments, the ratio of epidermal to dermal precursor cells is from about 1:3 to about 1:15, or alternatively, from about 1:5 to about 1:10, and increments within these ranges, e.g., or alternatively about 1:1, or alternatively about 1:2, or alternatively about 1:3, or alternatively about 1:4, or alternatively about 1:5, or alternatively about 1:6, or alternatively about 1:7, or alternatively about 1:8, or alternatively about 1:9, or alternatively about 1:10, or alternatively about 1:12, or alternatively about 1:13, or alternatively about 1:14, or alternatively outside this range, e.g., about 1:20 or alternatively about 1:50.

The compositions and methods can further comprise, or alternatively consists essentially of, or yet further consist of, admixing an effective amount of an agent inhibiting Bone Morphogenic Protein (BMP) signaling in the medium. The agent can be selected from the group consisting of dorsomorphin, noggin, chordin, gremlin, sclerostin, follistatin, and combinations thereof.

The composition and method can further comprise, or alternatively consists essentially of, or yet further consist of, admixing an effective amount of an agent promoting cell differentiation or growth. The agent can selected from the group consisting of Platelet Derived Growth Factor (PDGF), Vascular Endothelial Growth Factor (VEGF), Epithelial Growth Factor (EGF), TGF-, Fibroblast Growth Factor (FGF), insulin, transferrin, retinoid, and combinations thereof.

The composition and method can further comprise, or alternatively consists essentially of, or yet further consist of, admixing an effective amount of minoxidil, finasteride, or an agent enhancing cell growth.

In a yet further aspect, the composition and method can further comprise, or alternatively consist essentially of, or yet further consist of, seeding the cells in the medium onto a biocompatible scaffold. In one aspect, the biocompatible scaffold is dried or lyophilized prior to admixing with the cells in serum-free medium. In another aspect, the cells are seeded by passively contacting the cells with the scaffold at a temperature range from about 25° C. to about 40° C. for about 30 minutes to about 2 hours.

In one aspect or each of the above aspects, the skin precursor cells comprise, or alternatively consist essentially of, or yet further consist of, stem cells, epidermal precursor cells or dermal precursor cells. Stem cells, epidermal and/or dermal precursor cells can be of any appropriate type, e.g., an animal such as a mammal, including a human. Non-human animals include, for example, murine (such as rats or mice), canine, such as dogs, leporids, such as rabbits, equine, bovine, simian, livestock, sport animals, and pets. In one aspect, the cell species type is selected for compatibility with the host into which the composition is implanted, e.g., murine for a murine host and human for a human host.

Epidermal precursor cells can be isolated from animal or human keritoncytes and selected as described herein or in Fortunel et al. (2003) J. Cell Science 118:4043-4052. In one embodiment, the epidermal cells comprise keratinocyte stem cells, follicular papillae, sheath cells, non-stem cell keratinocytes, or any combination thereof.

Dermal precursor cells also can be isolated from non-human animals as described herein or from human sources as described in Medina et al. (2006) J. of Cellular Biochem. 98(1):174-84.

In a further aspect, adult or somatic stem cells can be utilized in the compositions of this invention. Typically, the cells are identified by the stem cells markers and can be isolated using the methods as described by, e.g., Reiisi (2009) In Vitro Cell Dev. Biol. Anim. 46(1): 54-59.

In one aspect the cells are allogeneic to the subject. In another aspect, the cells are autologous. In a further aspect, the cells are a mixture of allogeneic and autologous.

In a further aspect, the compositions comprises, or alternatively consists essentially of or yet further consists of a combination of dermal precursor cells, epidermal precursor cells, and stem cells, e.g. one or more of adult or somatic stem cells, embryonic stem cells and iPS cells. In some embodiments, the ratio of epidermal to dermal precursor cells is from about 1:3 to about 1:15; or alternatively from about 1:4 to about 1:12 and ratios within these ranges.

In a further aspect, the composition further comprises a detectable marker or label to monitor growth and differentiation of the cells. Examples of such include for example, luciferase under the control of a ubiquitin promoter, GFP, herpes simplex virus type 1 thymidine kinase (HSV-1 TK) under the control of a ubiquitin promoter and super-paramegnetic iron oxide (SPIO) nanoparticles. These systems are useful to detect teratoma formation or anomalous skin structures.

When the cells are in the scaffold, the concentration of the cells in the scaffold is from about 800,000 cells/mm³ to 1,500,000 cells/mm³. In some embodiments, the scaffold and cells are admixed by passively contacting the cells with the scaffold at a temperature range from about 25 to about 37° C. for about 30 minutes to 2 hours.

The compositions can alternatively contain an effective amount of differentiation or growth factor that promotes cell differentiation or growth. Non-limiting examples of such factors include agents that inhibit Born Morphogenic Protein (BMP) signaling, such as noggin (UniProt: Q13253) which can be produced using methods described in, e.g. McMahon et al. (1998) Genes & Development, 12:1438-52, chordin, gremlin, sclerostin, follistatin, and any combination thereof. Use of the terms such as “growth factors, cytokines, hormones” is to be exemplary. In one embodiment, the factor comprises Platelet Derived Growth Factor (PDGF) available from R&D Systems, Minneapolis, Minn., Vascular Endothelial Growth Factor (VEGF) available from Abcam, Cambridge, Mass., Epithelial Growth Factor (EGF) available from Abcam, Cambridge, Mass., TGF-available from Abcam, Cambridge, Mass., Fibroblast Growth Factor (FGF), insulin available from Abcam, Cambridge, Mass., transferrin, retinoid, or any combination thereof. In another embodiment, the composition is suitable for culturing mammalian epidermal cells and therefore can comprise cell culture medium as known to those of skill in the art, e.g., without limitation serum-free medium commercially available from Invitrogen (Carlsbad, Calif.). Additional components are optionally added to the composition, that include, but are not limited to antibiotics, albumin, amino acids, and other components known to the art for the culture of cells. Additionally, components optionally are added to enhance the differentiation process. Effective amounts of the differentiation and/or growth factors can be empirically determined by those of skill in the art. It is appreciated that such amounts will vary with the source of the cells, the ultimate composition (differentiated cell type(s)) desired after culturing or the differentiation of the cells and/or growth factors and the ultimate utility for the composition. An effective amount for an in vitro screen will not necessarily be the same as when the composition is to be administered to an animal such as a human patient.

The compositions can alternatively contain an effective amount of minoxidil (commercially available under the trademark “Rogaine” (Pharmacia & Upjohn Company)), finasteride, or other agent that enhances hair growth.

The invention also provides compositions comprising the suitable medium, e.g., serum-free medium described herein, wherein the medium comprises reduced concentrations of one or more factors that modulate cell growth. In one embodiment, the factor comprises PDGF, VEGF, EGF, TGF-, FGF, insulin, transferrin, retinoid, or any combination thereof. In another embodiment, the medium is suitable for culturing mammalian (e.g., murine, rat or human) epidermal cells. In another embodiment, the culturing comprises cell differentiation. In one embodiment, the epidermal cells comprise keratinocyte stem cells, follicular papillae, sheath cells, non-stem cell keratinocytes, or any combination thereof.

In the above embodiments, the concentration of cells in the scaffold is from about 800,000 cells/mm³ to about 1,500,000 cells/mm³. In some aspects, the concentration of cells in the scaffold is equal to or greater than about 10,000 cells/mm³, or alternatively is equal to or greater than about 50,000 cells/mm³, or alternatively is equal to or greater than about 100,000 cells/mm³, or alternatively is equal to or greater than about 200,000 cells/mm³, or alternatively is equal to or greater than about 300,000 cells/mm³, or alternatively is equal to or greater than about 400,000 cells/mm³, or alternatively is equal to or greater than about 500,000 cells/mm³, or alternatively is equal to or greater than about 600,000 cells/mm³, or alternatively is equal to or greater than about 700,000 cells/mm³, or alternatively is equal to or greater than about 800,000 cells/mm³, or alternatively is equal to or greater than about 900,000 cells/mm³, or alternatively is equal to or greater than about 1,000,000 cells/mm³. In some aspects, the concentration of cells in the scaffold is equal to or less than about 800,000 cells/mm³, or alternatively is equal to or less than about 900,000 cells/mm³, or alternatively is equal to or less than about 1,000,000 cells/mm³, or alternatively is equal to or less than about 1,100,000 cells/mm³, or alternatively is equal to or less than about 1,200,000 cells/mm³, or alternatively is equal to or less than about 1,300,000 cells/mm³, or alternatively is equal to or less than about 1,400,000 cells/mm³, or alternatively is equal to or less than about 1,500,000 cells/mm³, or alternatively is equal to or less than about 1,600,000 cells/mm³, or alternatively is equal to or less than about 1,700,000 cells/mm³, or alternatively is equal to or less than about 1,800,000 cells/mm³, or alternatively is equal to or less than about 1,900,000 cells/mm³, or alternatively is equal to or less than about 2,000,000 cells/mm³, or alternatively is equal to or less than about 5,000,000 or alternatively is equal to or less than about 10,000,000 cells/mm³. In one aspect, the skin precursor cells comprise epidermal and dermal precursor cells.

In some embodiments, the ratio of epidermal to dermal precursor cells is from about 1:3 to about 1:15, or alternatively, from about 1:5 to about 1:10, and increments within these ranges, e.g., or alternatively about 1:1, or alternatively about 1:2, or alternatively about 1:3, or alternatively about 1:4, or alternatively about 1:5, or alternatively about 1:6, or alternatively about 1:7, or alternatively about 1:8, or alternatively about 1:9, or alternatively about 1:10, or alternatively about 1:12, or alternatively about 1:13, or alternatively about 1:14, or alternatively outside this range, e.g., about 1:20 or alternatively about 1:50.

In some aspects, the composition can further comprise, or alternatively consist essentially of, or yet further consist of, an effective amount of a suitable carrier and/or a growth or differentiation factor. In one aspect, the factor is selected from the group consisting of noggin, chordin, gremlin, sclerostin and follistatin and combinations thereof. In another aspect, the factor is selected from the group consisting of Platelet Derived Growth Factor (PDGF), Vascular Endothelial Growth Factor (VEGF), Epithelial Growth Factor (EGF), TGF-, Fibroblast Growth Factor (FGF), insulin, transferrin, retinoid and combinations thereof.

In a further aspect, the composition further comprises a detectable marker or label to monitor growth and differentiation of the cells. Examples of such include for example, luciferase under the control of a ubiquitin promoter, GFP, herpes simplex virus type 1 thymidine kinase (HSV-1 TK) under the control of a ubiquitin promoter and super-paramegnetic iron oxide (SPIO) nanoparticles. These systems are useful to detect teratoma formation or anomalous skin structures.

In one aspect of this invention, the composition may be prepared by admixing an effective amount of isolated skin precursor cells in a suitable medium such as serum-free medium and optionally, a biocompatible scaffold, under conditions that favor the incorporation of the cells into the biocompatible scaffold when it is included. In some embodiments, suitable serum-free media can support the maintenance and expansion of stem cells or precursor cells and various types of serum-free media are commercially available from vendors. For examples, StemSpan® SFEM and StemSpan® H3000 are available from STEMCELL Technologies, Vancouver, BC, Canada. In some embodiments, the concentration of the cells to be admixed with the scaffold is an amount that will produce a concentration in the medium from about 800,000 cells/mm³ to about 1,500,000 cells/mm³.

In some embodiments, the scaffold and cells are admixed by passively contacting the cells with the scaffold at a temperature range from about 25 to about 37° C. for about 30 minutes to about 2 hours. In one embodiment, the media containing the cells is merely placed on a surface of the scaffold.

In some embodiments, different ratios between the epidermal and dermal populations can be used to make the composition. A ratio of epidermal and dermal precursor cells between about 1:3 to about 1:15; or alternatively from about 1:4 to about 1:12; or alternatively about 1:5 to about 1:10; 1:5 and about 1:10 and ratios in between these ranges, can be used to generate good pilosebaceous units. A combination of aged epidermal cells and newborn dermal cells, or a combination of newborn epidermal cells and aged dermal cells may not give rise to good hair growth. However, it has been noted that a replacement of newborn epidermal cells with aged epidermal cells had a lesser effect than a replacement of newborn dermal cells with aged dermal cells. Precursor cells can also be used to generate good hair growth. A combination of positive precursor cells and whole skin (WT) cells can lead to fair hair growth as well. While the use of Integra Matrix produced good hair growth, use of other scaffolds can result in good hair growth too.

This invention further provides a dermal patch comprising the compositions as noted above in combination with a dressing. A “dressing” refers to an overlay adjunct used by a mammal for application to a wound to promote healing and/or prevent further harm. A dressing may further comprise a bandage, which is primarily used to hold a dressing in place. In one aspect, a dressing can control the moisture content, protect the wound from infection, remove slough, or maintain the optimum pH or temperature to encourage healing. Non-limiting examples of dressings include a silicone protective layer or sheet, a collagen sheet, a plastic sheet or a latex sheet. In one aspect, the dressing is sterile. In a further aspect, the surface area of the dressing includes the entire area of the patch and extends beyond the periphery of the dermal patch and may optionally include an adhesive layer or coating around the periphery of the dressing but excluding the area of the patch. The adhesive coating or layer serves to secure the dermal patch to the situs of application. In a further aspect, the adhesive coating may exclude or include the area of the patch and if the adhesive coating includes the area of patch then the adhesive coating is irreversibly attached to the patch, or the adhesive coating can be reversible. In a yet further aspect, the dermal patch is stably attached to the dressing. In a yet further aspect, the dermal patch is removably attached to the dressing to allow for removing the patch overlay without removing the underlying patch.

Also provided by this invention is a method for generating pilosebaceous units in a physiological plane in a mammal in need thereof, comprising implanting the composition of the invention into the dermal layer of the subject such as a mammal under conditions that favor implantation of the composition into the dermis of the mammal. As used herein, mammals include, but are not limited to, murines, rats, simians, bovines, canines, humans, farm animals, sport animals and pets.

### Biocompatible Scaffolds

For the purpose of illustration only, examples of biocompatible scaffolds for use in this invention include, but are not limited to the porous and/or biodegradable and/or biocompatible scaffold as described in U.S. Pat. No. 4,947,840, col. 2, line 27 to col. 5, line 10, incorporated herein by reference in its entirety. In some other embodiments, a biocompatible scaffold is a dermal substitute consisting of amnion and biodegradable polymer as described in U.S. Patent Application Publication No. US 2005/0107876, paragraphs 28 to 64. In some other embodiments, a biocompatible scaffold is a single or double density biopolymer foam as described in International Patent Application Publication No. WO 98/22154, page 5, line 32 to page 23, line 33. In some other embodiments, a biocompatible scaffold is a gel-matrix-cells integrated system as described in International Patent Application Publication No. WO 2007/141028, page 13, line 1 to page 21, line 2. In some other embodiments, a biocompatible scaffold is a biomechanical implant as described in International Patent Application Publication No. WO 98/40111, page 7, line 13 to page 19, line 9.

In some embodiments, a biocompatible scaffold is a biocompatible nanofiber matrix as described in Venugopal et al. (2005) Tissue Engineering 11(5/6):847-54.

Examples of commercially available biocompatible scaffolds include, but are not limited to, Alloderm dermal collagen matrix (LifeCell Corporation, Branchburg, N.J.), Dermagraft-TC woven bioabsorbable polymer (polyglycolic and polylactic acids) membrane (Advanced Tissue Sciences, La Jolla, Calif.), Dermalogen human dermal collagen matrix (Collagenesis, Beverly, Mass.), Integra Bilayer Matrix Wound Dressing (Integra Life Sciences Corporation, Plainsboro, N.J.) and Fibrin Sealant Tisseel VH fibrin glue mixture (Baxter Health, Deerfield, Ill.). In some embodiments, the biocompatible scaffold can be type I collagen or silicon cell culture insert which are commercially available (e.g. Falcon™ Cell Culture Insert from BD Biosciences, San Jose, Calif.).

To make the composition, one admixes an effective amount of skin precursor cells in serum-free medium and a biocompatible scaffold, under conditions that favor the incorporation of the cells into the biocompatible scaffold. In another aspect, the cells are prepared as described above in the serum free medium and then seeded into the scaffold. In one aspect, the resulted concentration of cells in the scaffold is from about 800,000 cells/mm³ to about 1,500,000 cells/mm³. In some aspects, the concentration of cells in the scaffold is equal to or greater than about 10,000 cells/mm³, or alternatively is equal to or greater than about 50,000 cells/mm³, or alternatively is equal to or greater than about 100,000 cells/mm³, or alternatively is equal to or greater than about 200,000 cells/mm³, or alternatively is equal to or greater than about 300,000 cells/mm³, or alternatively is equal to or greater than about 400,000 cells/mm³, or alternatively is equal to or greater than about 500,000 cells/mm³, or alternatively is equal to or greater than about 600,000 cells/mm³, or alternatively is equal to or greater than about 700,000 cells/mm³, or alternatively is equal to or greater than about 800,000 cells/mm³, or alternatively is equal to or greater than about 900,000 cells/mm³, or alternatively is equal to or greater than about 1,000,000 cells/mm³. In some aspects, the concentration of cells in the scaffold is equal to or less than about 800,000 cells/mm³, or alternatively is equal to or less than about 900,000 cells/mm³, or alternatively is equal to or less than about 1,000,000 cells/mm³, or alternatively is equal to or less than about 1,100,000 cells/mm³, or alternatively is equal to or less than about 1,200,000 cells/mm³, or alternatively is equal to or less than about 1,300,000 cells/mm³, or alternatively is equal to or less than about 1,400,000 cells/mm³, or alternatively is equal to or less than about 1,500,000 cells/mm³, or alternatively is equal to or less than about 1,600,000 cells/mm³, or alternatively is equal to or less than about 1,700,000 cells/mm³, or alternatively is equal to or less than about 1,800,000 cells/mm³, or alternatively is equal to or less than about 1,900,000 cells/mm³, or alternatively is equal to or less than about 2,000,000 cells/mm³, or alternatively is equal to or less than about 5,000,000 cells/mm³ or alternatively is equal to or less than about 10,000,000 cells/mm³.

In one aspect, the skin precursor cells comprise epidermal and dermal precursor cells. In some embodiments, the ratio of epidermal to dermal precursor cells is from about 1:3 to about 1:15, or alternatively, from about 1:5 to about 1:10, and increments within these ranges, e.g., or alternatively about 1:1, or alternatively about 1:2, or alternatively about 1:3, or alternatively about 1:4, or alternatively about 1:5, or alternatively about 1:6, or alternatively about 1:7, or alternatively about 1:8, or alternatively about 1:9, or alternatively about 1:10, or alternatively about 1:12, or alternatively about 1:13, or alternatively about 1:14, or alternatively outside this range, e.g., about 1:20 or alternatively about 1:50.

In another aspect, the admixing is performed by passively contacting the cells with the scaffold, such as by soaking the scaffold with the cell composition in a pharmaceutically acceptable carrier at a temperature of about 25° C. to about 37° C. In one aspect, the biocompatible scaffold is dried or lyophilized prior to admixing with the cells in serum-free medium. In a further aspect, the method comprises, or alternatively consists essentially of, or alternatively, consists of admixing an effective amount of a growth factor selected from the group consisting of Platelet Derived Growth Factor (PDGF), Vascular Endothelial Growth Factor (VEGF), Epithelial Growth Factor (EGF), TGF-, Fibroblast Growth Factor (FGF), insulin, transferrin, retinoid and combinations thereof. The additional agents can be combined with the scaffold and/or with the cells at the same time (concurrently) or after combination of the scaffold and cells, or prior to admixing the scaffold and cells.

### Skin Precursor Cell Sources

In one aspect, the skin precursor cells comprise dermal and epidermal precursor cells. In another aspect, the precursor cells comprise progenitor cells from adult skin or other tissues containing stem cells. In another aspect, the precursor cells can be adult or embryonic stem cells having the ability to differentiate into hair follicles under appropriate culturing or growth conditions that are present in the micro- or macro-environment (see e.g. Yu et al. (2006) Am. J. Pathol. 168(6):1979-88).

In some aspects, the skin precursor cells are embryonic stem (ES) cells. ES cells have the potential to develop into different cell types. Attempts have been made to guide them toward a particular lineage with selected medium conditions, activating endogenous transcriptional factors (Pera & Trounson (2004) Development 131(22):5515-25), transfecting cells with specific transcriptional factors (Muller et al. (2000) FASEB J. 14(15):2540-8), or co-culturing them with cells capable of lineage induction (Kawasaki et al. (2000) Neuron 28(1):31-40). Several successful methods can guide mouse ES cells toward a keratinocyte lineage (Aberdam (2004) Int. J. Dev. Biol. 48(2-3):203-236; luchi et al. (2006) Proc. Natl. Acad. Sci. U.S.A. 103:1792-1797; Coraux et al. (2003) Curr. Biol. 13(10):849-853; Ji et al. (2006) Tissue Eng. 12(4):665-679).

In some aspects, the skin precursor cells are cells isolated from human tissues. In one aspect, the skin precursor cells are foreskin cells isolated from young children. In another aspect, the precursor cells are from adult human tissues. In one of such aspects, the skin precursor cells are cells isolated from the patient in need of the treatment. One of the risks in using stem cells is immunologic rejection, which can be alleviated by using a patient's own cells. It is valuable to isolate or convert adult cells to multi-potential skin stem cells.

In some embodiments, the skin precursor cells can be isolated from adult mammalian skin, such as Skin-derived Precursors (SKP) cells (Toma et al. (2005) Stem Cells, 23(6):727-737), or those from adipose tissue or bone marrow. These adult cells can be converted or differentiated into hair forming cells with procedures described in e.g. Hunt et al. (2008) Stem Cells 26(1):163-72. In another aspect, small molecules such as those targeting genes in the BMP pathway and Wnt pathway may be used to convert adult skin cells into progenitor cells (see e.g. Plikus et al. (2008) Nature 451(17):340-345).

In some aspects, methods or compositions known in the art can be used to induce hair forming ability from cells. In one aspect, acellular matrix is used. Acellular matrix is prepared from mammalian tissues (Schedin et al. (2004) Oncogene. 23(9):1766-79; Potapova et al. (2008) Am. J. Physiol. Heart Circ. Physiol. 295(6):H2257-63). Candidate cells are seeded in an acellular matrix derived from E13 mouse skin which has strong hair inducing ability. Hair follicles can induced from proper candidate cells.

In another aspect, small molecules and growth factors are used to induce the hair forming capability in cells. These cells are pretreated with growth factors or small molecules. Selection of candidate growth factors or small molecules is based on literature or microarray gene profiling analysis. They can be tested with methods described herein.

In another aspect, the skin precursor cells are Induced Pluripotent Stem (iPS) cells generated from cells isolated from adult tissues such as the skin by altering the transcription profile in the adult cells (see Takahashi et al. (2007) Cell 131(5):861-872 and Yu et al. (2007) Science 318(5858):1917-1920). These iPS cells can be converted to hair forming dermal papilla when they are incubated with stem cells with hair forming epidermis. The iPS cells can be converted to hair forming epidermis when they are incubated with stem cells with hair forming dermis or cell free matrix. In another aspect, the skin precursor cells comprise human adult keratinocytes and fibroblast cells.

Sources of skin precursor cells can be tested experimentally. In one aspect, multi-potential epidermal or dermal stem cells can be tested experimentally. For example, newborn mouse skin cells can serve as the positive control. Human or mouse or other types of mammalian epidermal stem cell candidates are tested in combination with newborn mouse dermal cells. Human or mouse or other types of mammalian dermal stem cell candidates are tested in combination with newborn mouse epidermal cells. Candidate cells are evaluated with a three-tier assay system with a higher throughput type screening first, and then with two of the more rigorous tests for hair forming ability. Tier (i), Mixed aggregate assay. In this assay, tested cells are mixed and cultured in shaking gassed flasks. Cells interact and differentiation genes are induced when right interactions occur. While cells sort to a certain extent, they remain disorganized. This assay is good for high throughput screening of cell interactions. This assay is based on the early work of Moscona (1980) Prog Clin Biol Res. 42:171-88, has been successfully used by the inventors as described in Grumet et al. (1984) Proc Natl Acad Sci USA 81(24):7989-7993, and a recent application of this principle to hair differentiation is reported in Havlickova et al. (2008) J Invest Dermatol. [Epub 2008 Aug. 26]. The ability of candidate cells to express hair follicle differentiation genes are tested with gene markers. The markers are screened by RT-PCR or immuno-staining or other technologies known in the art. Tier (ii) Patch assay (as described in Zheng et al. (2005) J. Invest. Dermatol. 124(5):867-876). In this assay, dermal and epidermal cells are mixed in a high density suspension and injected subcutaneously. This assay is used to test the ability of cells to form a hair follicle structure. The number of hair filaments formed can be quantified. However, hair cysts form on the underside of the skin thus the score has to be made on the underside of the skin exposed. Tier (iii) Planar hair forming assay, as disclosed in the specification. This assay evaluates the topology of the whole hair follicle population to see if they are properly or physiologically arranged.

In one aspect of this invention, the composition may be prepared by admixing an effective amount of isolated skin precursor cells in serum-free medium or other pharmaceutically acceptable carrier and a biocompatible scaffold, under conditions that favor the incorporation of the cells into the biocompatible scaffold. In some embodiments, the scaffold and cells are admixed by passively contacting the cells with the scaffold at a temperature range from about 25 to about 37° C. for about 30 minutes to about 2 hours. Passive is just applying to the surface of the scaffold. Additional agents, as describe above, can be further combined with the cells and scaffold.

Another aspect of this invention provides a method for generating pilosebaceous units in a physiological plane in a mammal in need thereof, comprising implanting the composition of this invention into the dermal layer of the mammal under conditions that favor implantation of the composition into the dermis of the mammal. In one aspect, the resulted concentration of cells in the scaffold is from about 800,000 cells/mm³ to about 1,500,000 cells/mm³. In some aspects, the concentration of cells in the scaffold is equal to or greater than about 10,000 cells/mm³, or alternatively is equal to or greater than about 50,000 cells/mm³, alternatively is equal to or greater than about 100,000 cells/mm³, alternatively is equal to or greater than about 200,000 cells/mm³, alternatively is equal to or greater than about 300,000 cells/mm³, alternatively is equal to or greater than about 400,000 cells/mm³, alternatively is equal to or greater than about 500,000 cells/mm³, alternatively is equal to or greater than about 600,000 cells/mm³, alternatively is equal to or greater than about 700,000 cells/mm³, alternatively is equal to or greater than about 800,000 cells/mm³, alternatively is equal to or greater than about 900,000 cells/mm³, or alternatively is equal to or greater than about 1,000,000 cells/mm³. In some aspects, the concentration of cells in the scaffold is equal to or less than about 800,000 cells/mm³, or alternatively is equal to or less than about 900,000 cells/mm³, or alternatively is equal to or less than about 1,000,000 cells/mm³, or alternatively is equal to or less than about 1,100,000 cells/mm³, or alternatively is equal to or less than about 1,200,000 cells/mm³, or alternatively is equal to or less than about 1,300,000 cells/mm³, or alternatively is equal to or less than about 1,400,000 cells/mm³, or alternatively is equal to or less than about 1,500,000 cells/mm³, or alternatively is equal to or less than about 1,600,000 cells/mm³, or alternatively is equal to or less than about 1,700,000 cells/mm³, or alternatively is equal to or less than about 1,800,000 cells/mm³, or alternatively is equal to or less than about 1,900,000 cells/mm³, or alternatively is equal to or less than about 2,000,000 cells/mm³, or alternatively is equal to or less than about 5,000,000 or alternatively is equal to or less than about 10,000,000 cells/mm³.

In one aspect, the skin precursor cells comprise epidermal and dermal precursor cells. In some embodiments, the ratio of epidermal to dermal precursor cells is from about 1:3 to about 1:15, or alternatively, from about 1:5 to about 1:10, and increments within these ranges, e.g., or alternatively about 1:1, or alternatively about 1:2, or alternatively about 1:3, or alternatively about 1:4, or alternatively about 1:5, or alternatively about 1:6, or alternatively about 1:7, or alternatively about 1:8, or alternatively about 1:9, or alternatively about 1:10, or alternatively about 1:12, or alternatively about 1:13, or alternatively about 1:14, or alternatively outside this range, e.g., about 1:20 or alternatively about 1:50.

In some embodiments, the conditions that favor implantation of the composition into the dermis of the mammal comprise, or alternatively consist essentially of, or yet further consists of applying suitable pressure to maintain contact between the composition and the muscle or subcutaneous fat of the mammal for at least 3 days. In some embodiments, the conditions that favor implantation of the composition into the dermis of the mammal comprise applying a dressing on top of the composition. In some embodiments, the dermal layer of the mammal was pretreated with an effective amount of an agent that inhibits Bone Morphogenic Protein (BMP) signaling. In one aspect of the embodiments, the agent is selected from the group consisting of dorsomorphin, noggin, chordin, gremlin, sclerostin and follistatin and combinations thereof.

In a further aspect, the cells further contain a detectable label that can be used to monitor the growth and differentiation of the cells in the subject.

In another aspect, optical methods can be used to monitor the growth and differentiation of the cells. Stem cells can be transduced using the lentivirus under the control of a constitutively active ubiquitin promoter driving the expression of luciferase. Animals can be anaesthetized, injected with D-luciferin and bioluminescence imaging can be performed in vivo using a Xenogen IVIS 200 System cooled CCD camera (Cheng et al. (2006) Bioconjug. Chem. 17:662-669; Love et al. (2007) J. Nucl. Med. 48(12):2011-20). Bioluminescence imaging can be used to check if these stem cells stay in the skin, close to where they were injected, or if they become diffuse, invasive, or distributed all over the body. It can also be checked if these cell products stay organized or start to become disorganized. Although the resolution of luciferase imaging is 1-2 mmm in vivo, the technique is sensitive, less costly, and has a higher temporal resolution (milliseconds) (Miller (2004) Adv. Drug Deliv. Rev. 56(12):1811-24) than other techniques. For in vivo detection of organization, GFP can be used. ES cells can be made to express GFP constitutively. Animals can also be engineered to express GFP constitutively. Transplantation of these cells onto a GFP negative host will allow one to visualize the organization of GFP positive cells using fluorescent microscopy (e.g. Leica Z16 APO fluorescent microscope). While the resolution of fluorescent imaging is much better, the light penetration is not good.

In a further aspect, micro PET/CT can be used for long-term tracking. To create a positron emission tomography (PET) reporter gene system, stem cells can be transduced with Herpes Simplex virus type-1 thymidine kinase (HSV-TK1) under the control of the constitutively active ubiquitin promoter. PET scans of stem cells preloaded with ¹⁸F-FDG or ⁶⁴Cu-PTSM provide imaging over only a period of days, because of the loss of signal from radioactive decay. HSV-TK1 high affinity PET radiotracer 9-[4-[¹⁸F]fluoro-3-(hydroxymethyl)butyl]guanine ([¹⁸F]FHBG) is also appropriate. PET scans with [¹⁸F]FHBG allows the flexibility to monitor stem cells over a span of several weeks through multiple time points. In addition, images can be acquired in conjunction with bioluminescence imaging Concorde Microsystems microPET R4 can be used, immediately followed by a CT scan using the Siemens Inveon microCT to produce co-registered PET/CT images. The PET data can then be reconstructed using the Maximum a Posteriori image (MAP) reconstruction, to provide higher spatial resolution PET images. The PET information acquired on the microPET can be co-registered with the CT data to provide the combination of stem cell location (PET data) layered on an anatomical reference image (CT data).

In yet another aspect, ultrasound can be used for tracking the cells. For ultrasound imaging, the skin is first shaved (not plucked) to avoid any damage to the hair follicles. The skin is then covered with aquasonic gel to facilitate contact of the ultrasound probe. Images are videotaped to produce real time movies of the skin. The overall architecture can be visualized.

### Therapeutic and Diagnostic Utilities

In one aspect, the invention provides a method for generating pilosebaceous units in a physiological plane in a mammal in need thereof, comprising, or alternatively consisting essentially of, or yet further consisting of implanting the composition of the invention into the dermal layer of the mammal under conditions that favor implantation of the composition into the dermis of the mammal. In one aspect, the composition replaces the entire skin within the area. In another aspect, the epidermis and all of the dermis are replaced by the composition. In yet another aspect, the epidermis and part of the dermis are replaced by the composition. One can determine when the method has been accomplished by noting the growth of hair or formation of pilosebaceous units in a topical plane in the mammal.

In another aspect, the invention provides a method for preparing pilosebaceous units in a physiological plane, comprising admixing skin precursor cells and a medium, wherein the concentration of skin precursor cells present in the medium is greater than about 1×10⁷ cells per milliliter of medium. In some embodiments, the concentration of skin precursor cells present in the medium is less than about 1×10⁸ cells per milliliter of medium. Yet in some embodiments, the concentration of skin precursor cells present in the medium is from about 2×10⁷ cells per milliliter of medium, or alternatively about 3×10⁷ cells per milliliter of medium, or alternatively about 4×10⁷ cells per milliliter of medium, or alternatively about 5×10⁷ cells per milliliter of medium to about 6×10⁷ cells per milliliter of medium, or alternatively about 7×10⁷ cells per milliliter of medium, about 8×10⁷ cells per milliliter of medium, or alternatively about 9×10⁷ cells per milliliter of medium, or alternatively about 1×10⁸ cells per milliliter of medium.

The skin precursor cells in the medium are comprised, or alternatively consisting essentially of, or yet further consisting of dermal precursor cells and epidermal precursor cells.

In some embodiments, the ratio of epidermal to dermal precursor cells is from about 1:3 to about 1:15, or alternatively, from about 1:5 to about 1:10, and increments within these ranges, e.g., or alternatively about 1:1, or alternatively about 1:2, or alternatively about 1:3, or alternatively about 1:4, or alternatively about 1:5, or alternatively about 1:6, or alternatively about 1:7, or alternatively about 1:8, or alternatively about 1:9, or alternatively about 1:10, or alternatively about 1:12, or alternatively about 1:13, or alternatively about 1:14, or alternatively outside this range, e.g., about 1:20 or alternatively about 1:50.

In some aspects, the composition can further comprise, or alternatively consist essentially of, or yet further consist of, an effective amount of a suitable carrier and/or a growth or differentiation factor. In one aspect, the factor is one or more of noggin, chordin, gremlin, sclerostin or follistatin. In another aspect, the factor is one or more of Platelet Derived Growth Factor (PDGF), Vascular Endothelial Growth Factor (VEGF), Epithelial Growth Factor (EGF), TGF-, Fibroblast Growth Factor (FGF), insulin, transferring or retinoid.

The skin precursor cells and the medium are admixed on any surface or within a container suitable for cell culture such as a cell culture insert as described herein. The size and shape of the surface or container is unlimited. In some embodiments, the container has a volume that is about 50 μl, or alternatively about 100 μl, or alternatively about 200 μl, or alternatively about 500 μl, or alternatively about 1 ml or more. The shape of the container is non-limiting. In some embodiments, the container is round, or alternatively square, or alternatively adopts the shape of the intended implant.

Suitable medium includes, without limitation, 1:1 DMEM/F12 with no serum. The cell slurries that form after the skin precursor cells and the mediums are mixed and allowed to settle down for about 1 to about 2 hours in an incubator set at a temperature of about 37° C. before grafting onto the host. The cell slurry is then can then be grafted onto the host. In transplantation, the cell slurry can be placed under a piece of epithelial sheet. The typical size is about 1.5 cm² although any range of up to about 2.5 mm in diameter or about 5 by 40 mm will also suffice.

Suitable membranes for the slurry include, without limitation, Integra™ and Falcon™ tissue culture insert. The Integra™ matrix is commercially available. The culture insert membrane (polyethylene terephthalate PET) is also commercially available from, e.g., BD Falcon, San Jose, Calif.

In some embodiments, the method further comprises, or alternatively consists essentially of, or yet further consists of, the step of overlaying an epithelial sheet on the admixed dermal precursor cells and the medium. The pilosebaceous units prepared by the method of the invention can be used to treat a condition in a mammalian subject in need of, which condition comprises hair loss or insufficient hair growth. In one aspect, the condition is alopecia. In another aspect, the condition is wound healing.

Mammals that may be suitably treated by this method include, but are not limited to those described as “subjects” herein. It is apparent to those skilled in the art that the cell source for therapeutic use should match or closely match the species into which cells and matrix are implanted. For example, when the method is practiced a human patient, the cell source should be human as well. However, when the purpose of the invention is to screen agents that can modulate the formation of pilosebaceous units in a topical plane, it is not necessary that the source of cells be identical to the subject being treated. It is conceivable that human cell sources may be implanted into mice (nude mice as shown below) and then agents are contacted with the implant either by incorporation into the matrix or alternatively by subsequent administration to the implanted cells and growth is monitored. The test agents can be compared to known agents that modulate hair growth, for example noggin or Minoxidol™ to determine if they are candidate leads for further development. This is a fast and simple clinically relevant animal model for high-throughput screening of various test agents.

In yet another aspect, the conditions that favor implantation of the composition or the scaffold into the dermis of the mammal comprises suitable pressure to maintain contact between the composition and the muscle or subcutaneous fat of the mammal for at least 3 days or alternatively at least 5 days, or yet further at least 7 days. In one aspect, pressure is maintained by covering the implant with a silicone covering for an effective amount of time.

In yet another aspect, the composition of this invention can be used to treat a condition in a mammalian subject in need of, which condition comprises hair loss or insufficient hair growth. In one aspect, the condition is alopecia. In another aspect, the condition is wound healing.

The agents and compositions of the present invention can be used in the manufacture of medicaments and for the treatment of humans and other animals as described and exemplified herein.

This invention also provides a non-human animal model to screen for agents that modulate the growth of hair in a physiological plane comprising, or alternatively consisting essentially of, or yet further consisting of a suitable subject having implanted into the tissue of the subject an effective amount of the cell and scaffold matrix as variously described above. The agent to be screened can be added to the scaffold/cell composition or alternatively, subsequently applied to the area of an animal or human that received the implant. The growth of hair and/or formation of pilosebaceous units is monitored and alternatively can be compared to a second animal receiving the same implant without the test agent or yet further or alternatively a third animal receiving a known agent such as noggin that modulates hair growth. Agents can either augment (support) or impede hair growth or the formation of pilosebaceous units. Alternatively, they may have substantially no therapeutic impact. However, agents that do modulate can be selected for further research and clinical development.

The agents, compositions and methods of the present invention in any of the above embodiments can be used in the manufacture of medicaments and for the treatment of humans and other animals by administration in accordance with conventional procedures, such as an active ingredient in pharmaceutical compositions.

### Kits

An aspect of the invention provides a kit for performing at least one therapeutic or diagnostic method of this invention comprising, or alternatively consisting essentially, or yet further consisting of an effective amount of a suitable biocompatible matrix and instructions for use which may include methods to isolate the precursor cells. In some embodiments, the pharmaceutically acceptable carrier in the kits is suitable for topical administration of the agent. In some embodiments, the pharmaceutically acceptable carrier further comprises a penetration or permeation enhancer.

Also provided are kits for administration of the compounds for treatment of disorders as described herein. Kits may further comprise suitable packaging and/or instructions for use of the cells and scaffold. Kits may also comprise a means for the delivery of the at least one agonist or antagonist and instructions for administration. Alternatively, the kit provides the compound and reagents to prepare a composition for administration. The composition can be in a dry or lyophilized form or in a solution, particularly a sterile solution. When the composition is in a dry form, the reagent may comprise a pharmaceutically acceptable diluent for preparing a liquid formulation. The kit may contain a device for administration or for dispensing the compositions, including, but not limited to, syringe, pipette, transdermal patch and/or microneedle.

The kits may include other therapeutic compounds for use in conjunction with the compounds described herein. These compounds can be provided in a separate form or mixed with the compounds of the present invention.

The kits will include appropriate instructions for preparation and administration of the composition, side effects of the compositions, and any other relevant information. The instructions can be in any suitable format, including, but not limited to, printed matter, videotape, computer readable disk, or optical disc.

In another aspect of the invention, kits for treating an individual who suffers from or is susceptible to the conditions described herein are provided, comprising a container comprising a dosage amount of a composition as disclosed herein, and instructions for use. The container can be any of those known in the art and appropriate for storage and delivery.

Kits may also be provided that contain sufficient dosages of the effective composition or compound to provide effective treatment for an individual for an extended period, such as a week, 2 weeks, 3, weeks, 4 weeks, 6 weeks, or 8 weeks or more.

The following examples are provide to illustrate select embodiments of the invention as disclosed and claimed herein.

### Experimental Examples

### Material and Methods:

Cell isolation. Multipotential skin precursor cells are currently obtained from neonatal mice using techniques as known in the art and disclosed, for example in WO 2010/059862. Briefly, neonatal mice are harvested shortly after birth (within the first 24 hours) and euthanized. The truncal skin is dissected with sharp forceps. Epidermis and dermis are separated by floating the skin in cold 0.25% trypsin solution overnight. Epidermal cells are then dissociated into a cell suspension by cutting into fine pieces and manual tituration with a serological pipet. Single epithelial cells are filtered through a 70 μm cell strainer to exclude cells of the stratum corneum. The dermal cells are individually dissociated using warm 0.35% collagenase solution for 40-50 minutes at 37° C. DNase I is added for 5 minutes at room temperature before manual tituration with a serological pipet. The collagenase and trypsin activities are stopped by washing cells in either trypsin inhibitor or medium containing a 10% fetal bovine serum. The cells are filtered through a 40 μm cell strainer to ensure single cell suspension and exclude as many of the pre-formed hair follicles as possible. Both sets of cells are then recombined in a ratio of 1 epidermal to 5-10 dermal cells and washed again DMEM:F12 (1:1). The cells are finally resuspended into 150-200 μl of DMEM:F12 (1:1) as a slurry.

### Cell Preparation and Grafting—Preparation of Cells for Transplantation

Newborn epidermal and dermal cells were used to generate hairs. They are known to be multi-potential skin stem cells. Dorsal skins are obtained from newborn mice, and processed into dissociated cells. After washing, epidermal and dermal cells are recombined in a defined ratio and are resuspended into a very small amount of medium (DMEM/F12 in the ratio of 1/1). This slurry of recombined cells is adjusted to be about 10-100 million cells/ml.

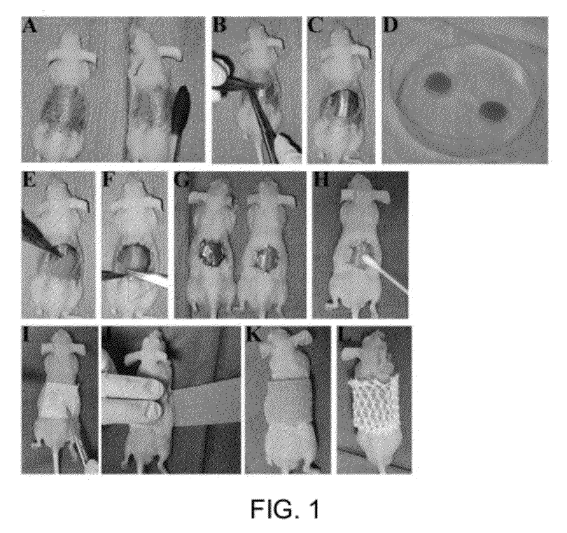

Dissociated epidermal and dermal stem cell candidates are prepared. They can be mixed in different ratios. The cell slurry was used as a drop with minimal amount of media. Usually, 150-200 μl cell suspension, with total cell number in the range of 2-20 million were pipetted onto a tissue cell culture insert. At this volume, cells can be held together as a drop by surface tension. If a larger area is desired, cells can also be walled by plastic well (a range from 5-15 mm in diameter have been used). The cell slurries are allowed to settle down for 1-2 hours in a 37° C. incubator, and excess liquids are allowed to dry (FIG. 1D). These conditions allow cells to generate a gel-like endogenous matrix. Freeform flap can also be generated by casting cells into certain shapes of plastic wells.

It is also possible to seed these cells into commercially available matrix. Previously, Matrigel™ or Integra™ were used were used to seed the cells. See, WO 2010/059862. When Integra™ matrix is used, the matrix is first rinsed several times with serum free medium. An advantage here is that it has a silicone supportive layer. The Integra™ matrix is cut into the desired size and shape, and blotted dry on sterile, non-stick gauze (with the silicone protective layer on the bottom). The cell slurry is then pipetted evenly onto the undersurface of the dry collagen matrix (Kremer, et al 2000). The Integra™ is about 1 mm thick. For each 1.5 cm² piece of Integra™ typically approximately 12 million epidermal cells and 60 million dermal cells in 200 μl of serum free medium, were used. For Matrigel™, it is processed in a similar way.

Similar to Zhang et al., 2005, Applicants have worked with different cell ratios, between the epidermal and dermal populations and determined with this technique, a ratio of 1:5-10 for epidermal:dermal cells is optimal.

### Grafting to the Host

Athymic nude, hairy SCID, or normal mice of the same inbred strain were prepared and draped with betadine solution under anesthesia. If using SCID mice, they were shaved first. (FIG. 1A). The intended area of skin to be grafted for hair bearing is excised in full thickness, leaving the musculature beneath undamaged (FIG. 1B, C). Bleeding, if any, is controlled with gentle pressure, and the tissue culture insert or collagen matrix, with cells on top or seeded inside, is flipped onto the wound. Cells are pressed against the wound bed with the insert membrane or the silicone protective layer level with the host skin epidermis (FIG. 1E). The membrane is sutured to the host skin (FIG. 1F, G). Sterile dressings are applied to provide constant pressure against the graft to the wound bed (FIG. 1H-L) so the graft has the best chance of being incorporated to the skin of the host.

Dressings are removed for inspection around days 8 post grafting. The sutures are removed and the protective silicone layer or insert can now be peeled off easily because the wounds have been re-epithelialized. Once dressings are removed, no special care of the animal is needed.

### Tissue Culture Inserts and Matrix Used

The Integra™ is commercially available and is from Integra LifeSciences (Plainsboro, N.J.). The culture insert membrane is made of polyethylene terephthalate and is from BD Falcon.

### Characterization of the Reconstituted Skin

Skin at different stages were removed. Paraffin sections were prepared.

H&E staining and immuno-staining were performed as described (Yeh et al., 2009). Antibody to NCAM is described by Chuong et al 1992 (Chuong et al., 1982). Antibody to keratin 14 is from Berkeley Antibody Company, Richmond, Calif. AE13 and Involucrin are from Abcam, San Francisco, Calif. Versican is from Millipore, Billerica, Mass.

### Wound Healing

Mice were anesthetized and small full thickness wound was produced as described (Yeh et al., 2009).

Hair Regeneration after Plucking

The regenerative hairs were stripped with warm paraffin or direct extraction using foreceps under anesthesia. Pictures were taken every 2-3 days to record hairs regeneration (Ma et al., 2003).

### Monitor of Hair Cycling

This was described in Plikus and Chuong, 2007 and Plikus et al., 2008. The regenerative hairs were clipped with a hair trimmer used in human barbershop. Pictures were taken every 2-3 days to record hairs growth. For this purpose, hairs have to be trimmed anew every time when a photo is to be taken.

### Results

### Characterization of Reconstituted Hairs

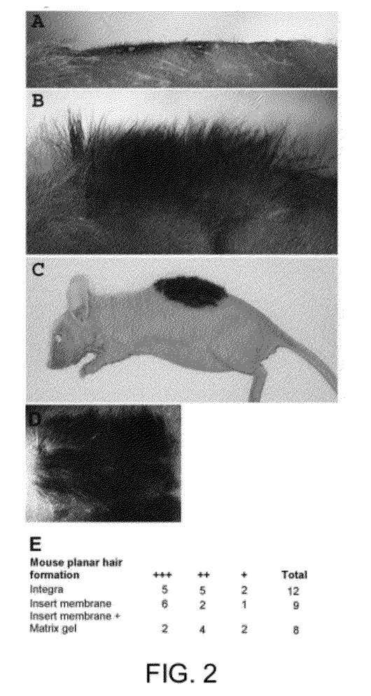

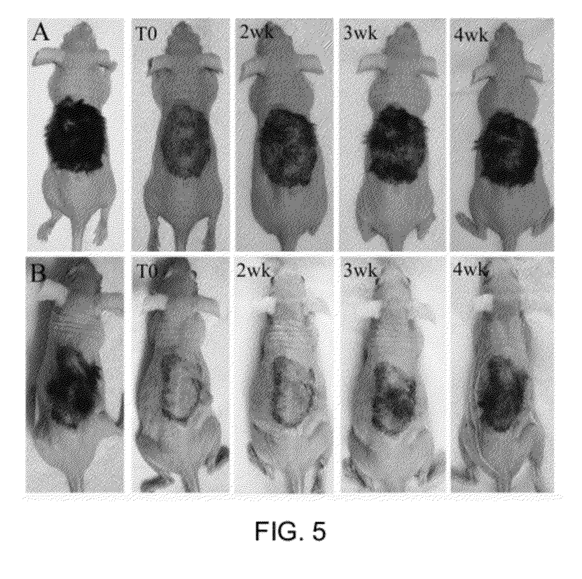

Hairs can be seen by the naked eye on the surface of the wound as early as 11-15 days post graft. Applicants have high reproducibility of hair formation (FIG. 2B). Here Applicants describe their arrangement in the gross view and molecular characterization on histological sections. The hair forms densely on the reconstituted skin and hairs are arranged on a plane and grow evenly in a cosmetically acceptable fashion (FIG. 2A to D). Under microscope, hair filament shows normal appearance. Apparent difference in preparation with tissue culture insert or with commercial matrix were not observed. Sometimes there is higher hair density closer to the wound margin, and tissue sections suggest that it is due to the accumulation of more cells around the sutures (FIG. 5). Controls include the use of Integra™ or Matrigel™ without cells. In these cases skin healed by wound contraction and re-epithelialization without new hair formation.

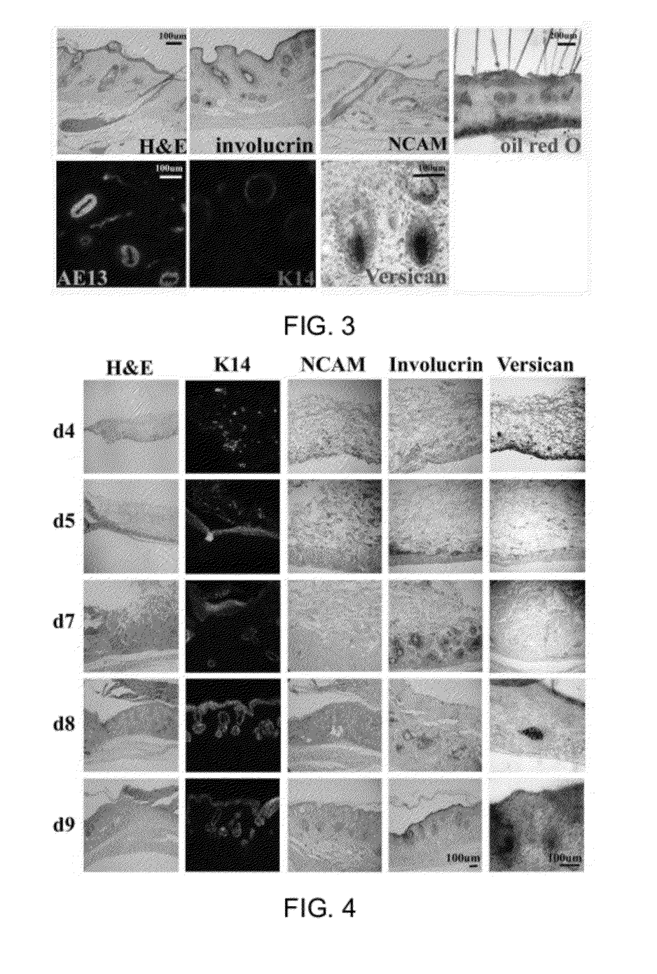

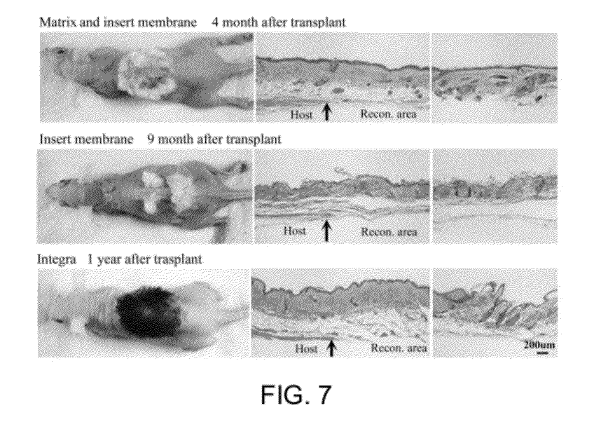

Histological sections of the skin at day 11 post graft show that normal layers of the skin, including the epidermis, hair follicles, sebaceous glands, subcutaneous adipose layer, dermis, etc. have been recreated (FIG. 3). They are arranged in the right architecture and each component is of the right size and shape. The epidermis is somewhat wrinkled at this stage, which becomes flat later (FIG. 7A). Applicants used several immuno-histochemical molecular markers to monitor their molecular differentiation (FIG. 3). K14 is present in the basal layer and the follicle heath. NCAM is mainly present in the dermal papilla. Versican is present strongly in the dermal papilla. Involcurin is present in the suprabasal keratinocytes. AE 13 is expressed in the inner root sheath. Oil red 0 is positive in the sebaceous gland and the subcutaneous adipose tissue. These staining patterns are similar to those reported for normal hair follicles.

To study the morphogenetic process that takes place in the formation of new hair follicles, Applicants prepared sections using specimens obtained from different post-operative days. The appearance of molecular markers were followed from day 4-9 post graft (FIG. 4). The molecular markers included keratin 14 (K14), neural cell adhesion molecules (NCAM), Involucrin and Versican. K14 is one of the type I keratin and hetero-dimer with keratin 5, a type II keratin. K14 and K5 are present on the proliferative basal keratinoyctes, and are usually used as a marker for proliferating basal epidermal layer. NCAM is a homophilic cell membrane glycoprotein that mediate cell-cell adhesion among several different cell types. Involucrin is a molecule involved in crosslinking epidermal envelope and usually used as a marker for differentiated epidermal keratinocytes. Versican is a large extracellular matrix proteoglycan that is present in a variety of human tissues. In hair follicles, it is enriched in dermal papilla and is used as a marker for growing phase dermal papilla.

K14: At day 4 post graft, K14 positive cells scatter around, without forming a sheet or aggregates, in the matrix. At day 5 post graft, the K14 positive cells start to coalesce and organize themselves into a basal epidermal layer. At day 8, hair pegs can be seen to invaginating into the newly generated dermis. NCAM: NCAM positive cells can be seen at day 4, distributed randomly in the matrix. At day 5, 7, the can be seen to be distributed in the dermis, and becomes enriched in dermal papilla at day 8, 9 when the morphology of hair follicles become clear. Involucrin: Positive cells appear at day 5 in the putative epidermis. At day 7, it is expressed in the suprabasal cells facing the cavity flanked by the invaginated epidermis. At day 9, it is limited to the suprabasal epidermis facing the outside. Versican: The expression is similar to NCAM. Positive cells are first distributed in the dermis randomly at day 4, and eventually and by day 8 have homed in to the dermal papilla.

Putting these together, Applicants determined that epidermal and dermal cells are randomly mixed together at day 4. Cell re-arrangement takes place in this 1 mm thick matrix. At day 5, epidermal cells sort themselves out and coalesce to form a basal layer first at the bottom of the matrix. They then “rise” from the base to the level of the air surface level (FIG. 4). Some transient cavities flanked by the invaginated epidermis can be observed at day 7, 8 and the epidermis eventually flattened out. At day 8, hair germs start to appear, which progress to hair peg stage at about day 9 and forming follicles at day 11.

Applicants note that the orientations of these hair follicles are all pointing toward the epidermal surface. The formed hair follicles point upward and hair filaments are able to protrude out to the surface of the skin. Applicants have never observed hair follicles pointing to the underside of the matrix or horizontally. When the matrix is overloaded with too many cells, occasionally there are some cysts remained in the dermis which failed to merge with epidermis. Some hairs can grow into these cavities, similar to those observed in the patch assay (Zheng et al., 2005)

Reconstituted Hairs Cycle Physiologically and can Regenerate after Plucking

One of the criteria to judge successful formation of engineered hair follicles is the ability of the follicle to cycle physiologically and to regenerate after plucking (Chuong et al., 2007). Here, Applicants examined their physiological cycling. Hairs were clipped short to allow the observation of anagen as described 6. Indeed, it can be seen that after hairs were clipped they can still grow and back to normal length in 2 months (FIG. 5A). The lengths of hair cycle vary in different animals, and also differ vary depending on age, sex, etc. In a two month old mouse, the estimated anagen is 28 days, and telogen is about 80 days. The hairs can cycle continuously up to more than one year.

Applicants then tested their regenerative ability after hairs are plucked. Applicants initially tested small patches of hair directly plucked individually by forceps. Once it was found that plucked hairs could regenerate, Applicants advanced to larger areas of hair removal to look at patterns of anagen. Hairs in the whole graft were plucked with warm wax. These hair follicles re-enter anagen in about 10 days, and pigmented anagen follicles are visible in 2 weeks. They continued to grow and reach the normal length in about 2 months (FIG. 5B).

Reconstituted Skin Lasts More than One Year and can Respond to Injury and Heal

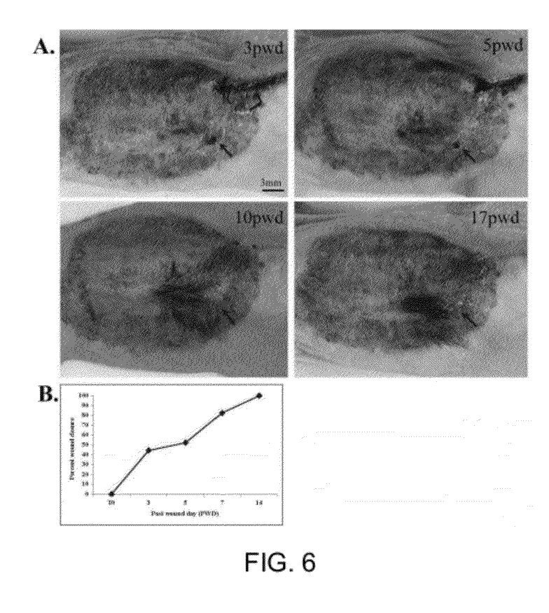

Applicants questioned the stability of the “reconstituted skin” and how they respond to a full thickness wound. Stability was tested this by making a 3 mm full thickness punched wound on the reconstituted skin (FIG. 6, arrow). The wound closed properly within 10 days, similar to that of the normal skin (Yeh et al., 2009; FIG. 6). Interestingly, hairs around the wound margin now grow faster, similar to the report that wound itself can stimulate the growth of existing hair follicles.

In terms of long term stability, Applicants produced reconstituted skin and are able to keep them up to 18 months. The hairs are still growing and cycling. Sections showed reconstituted skin can form normal appearing epidermis, dermis, and integrated with the host skin (FIG. 7). Hair follicles and subcutaneous adipose tissue are also observed.

However, the subcutaneous muscle layer is not reformed. There are no difference of skin quality or hair growth when tissue culture insert, Matrigel™ or Integra™ are used.

Applicants also transplant cells from constitutive GFP positive mice to the same strain of mice. A majority of grafted cells are seen to stay in the grafted region after 9 months, although the border is not sharp and cell mixing can be observed at the junction (FIG. 7B).

This also shows the ability of these newborn mice to form reconstituted skin does not have to do with the compromise immune ability of nude or SCID mice.

### Assays and Screens

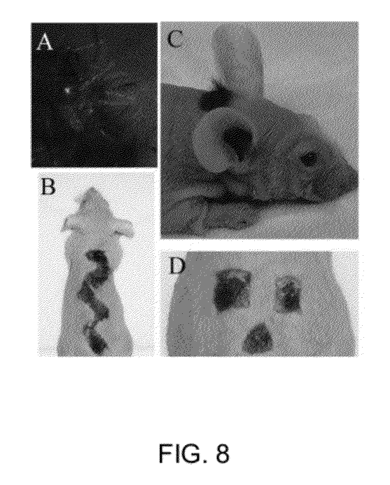

Applicants also tested if lenti-viruses could be used for this purpose as a transducer of candidate genes for reprogramming. Lentivirus carrying green fluorescent protein (GFP) was used to transduct newborn skin cells, either dermal or epidermal. As seen by GFP, the transduction efficiency was high and these cells also moved on to form “hairs normally (FIG. 8A). So they can be used to test if the lenti-mediated suppression or upregulation of certain genes are essential for new hair formation. On the other hand, adult keratinocytes or adult skin fibroblasts were used to replace one of the components. In both cases, there is no new hair formation. In this case, this procedure can serve as a platform for large scale screening of molecular candidates. Thus, the above methods are further modified by delivering the agent to be tested, by use of a viral vector such as a lentiviral vector.

For clinical applications, it would be useful to be able to accommodate irregular sizes and shapes of the wounds. Because the matrix, whether endogenously produced or exogenous matrix, is reasonably stiff, it can be also be shaped flexibly in free form as needed (FIGS. 8B through D). This can conceivably be made clinically useful in the reconstructive procedures of hair replacement of particularly shaped regions of hair growth (i.e. eyebrows). In terms of the size of the reconstituted skin, Applicants have made graft of about 500 mm² surface area on the mouse with successful hair growth. This is approximately 30% of a mouse's total body surface area. Additionally, a few of the mice have endured multiple grafts in different parts of their bodies. This procedure is also tolerable in multiple stages. For the potential use toward alopecia, it may be useful to make the region as small as possible. Grafts as small as 0.5 cm² have been made with successful hair formation. Thus the reconstituted skin can be made with flexibility in the shape and size.

In the age of tissue engineering, there is desire to reconstitute hair follicles from dissociated single cells (Stenn et al., 2005). The cells have to be multi-potential stem cells. The challenge is that these cells not only have to differentiate into different cell types, they have to be arranged with the proper organization. This is important at the level of intra-follicular organization, as well as in the arrangement of a population of follicles. The present disclosure describes an improved and simplified procedure that allows multi-potential skin precursor cells to form a large number of de novo hair follicles on a plane. These cells self-organize in a plane, forming a skin with a cosmetically acceptable appearance. Histologically, the reconstituted skin shows proper proportions and topological arrangements of different skin components including the hairs, sebaceous glands, dermis, subcutaneous adipose tissues, but not the muscle layer.

This work builds on earlier achievements by Lichti (Lichti et al., 1993 and Yeh et al. 2009) and Stenn's group (Zheng et al., 2005). Other investigators also have worked to produce high throughput assays for hair forming ability. Havlickova et al. 2009 have developed a way to evaluate the effect of different molecules on hair formation. However, the formed structures do not progress into real hair follicles. Different methods to manipulate the matrix and dermal cell aggregates have been developed to facilitate the formation of reconstituted skin and development of hairs with different levels of success (Nakao et al., 2007, Qiao et al., 2008, Powell et al., 2009 and Yen et al., 2010). The procedure of the present disclosure is advantageous in that it can be performed efficiently and on a large scale so that it can be used for a high throughput screening of molecules important for the formation of hair follicles.

It is important that the engineered hair follicles fulfill the definition of hair follicles. As such, the Applicant has earlier developed a definition of hair follicles that includes the concentric hair filament organization, proper hair follicle configuration, stem cell and transient amplifying cell topology that allows proximal-distal growth, the association of sebaceous glands, and the ability to save stem cells for repetitive cycling (Chuong et al., 2007). Here the reconstituted skin hair follicles were evaluated with these criteria. It is shown that the reconstituted hair follicles show the proper follicular organization, express hair differentiation markers, and contain sebaceous glands. The reconstituted hair cycles under physiological conditions. They also respond to injuries caused by hair plucking and wax stripping and regenerate properly. Further, the skin was injured with full thickness wounds. Skin healing can take place properly in a timely fashion. The reconstituted skin on the mice have been followed up which have been transplanted up to 18 months. Hair growth and cycling are still active. Thus, with multi-potential skin stem cells, this procedure provides an excellent platform for the formation of reconstituted skin.

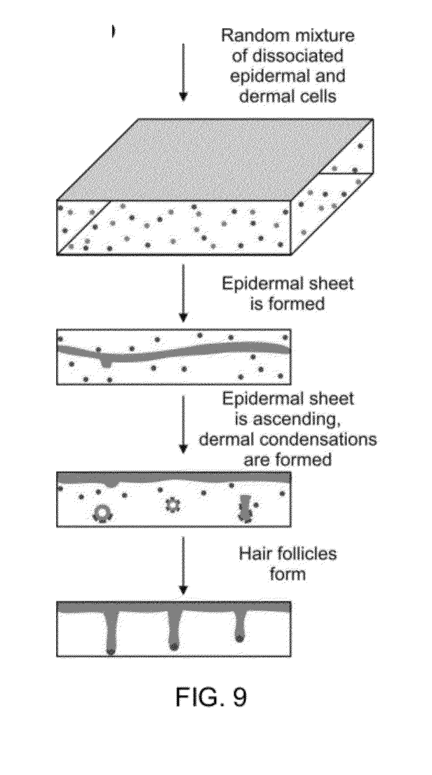

While the hair forming process parallels that in development, by analyzing the morphogenetic process in regeneration, several novel morphogenetic processes have been observed that do not occur in development. (1) The originally randomly distributed epidermal and dermal cells gradually sort themselves out. This may be explained by the more adhesive force among epidermal cells compared to the epidermal-dermal adhesiveness, or the dermal-dermal adhesiveness (Yen et al., 2010). (2) At day 5 after grafting, most epidermal cells are near the bottom of the graft. The second surprise is that these epidermal cells, in the form of aggregates or dissociated cells start to shift upward toward the air surface. During this process, some small epidermal cellular aggregates are seen in the process of coalescing as they rise. They become connected to the outer epidermis and flatten out. Eventually a flat epidermal layer lies on top of the dermal layer. (3) The ability of dermal cells to form periodically arranged dermal condensations adjacent to the epidermal cells. These dermal condensations and the adjacent epidermis then progress to become follicles (FIG. 9).

How these different processes occur at the cellular and molecular level constitute future challenge. For example, the mechanism for epidermal cells to form aggregates/cysts or flat configurations is most interesting. It probably involves changes of cell adhesion and mechanics. The mechanism by which this occurs and the establishment of correct apical-basal cellular polarity pose other mysteries. It is herein speculated that epidermal cells may be attracted by an oxygen gradient. For periodic pattering, the mouse dermal and epidermal cells delivered at this stage are probably still competent for morphogenesis, and can respond to the activators and inhibitors, leading to the periodic pattern formation of skin appendages (Maini et al., 2006). This process provides the possibility to modulate the size of hair germs by altering the concentration of these activator and inhibitor activities (Jiang et al., 1999). Thus this model provides a great opportunity to study the morphogenetic ability of stem cells during regeneration and reconstitution.

Molecular re-programming has now opened up possibilities to switch the fate of cells (Gurdon and Melton 2008). A similar strategy can be used to reprogram somatic cells to gain or lose the ability to form hairs. To achieve this potential in drug discovery, a simplified procedure to screen for small molecules or genes with a clear readout will be needed. High throughput screening of small molecules is possible. To pave the way for gene screening, the Applicant transduced these cells with lentivirus carrying GFP, and showed hairs can still form normally. Thus lentivirus can be used to over-express or suppress candidate genes that are involved in hair growth.

In summary, the present disclosure provides a simple one step procedure in which cells are able to self-organize and differentiate properly to generate reconstituted skins. A large number of pilosebaceous units are generated and distributed in a plane with a cosmetically acceptable arrangement. The graft can be made with flexibility into flexible shape and size. Thus, this procedure sets up a model for translational research and clinical applications can be implemented.

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

### REFERENCES

Chuong C M, Cotsarelis G, Stenn, K. (2007) Defining hair follicles in the age of stem cell bio-engineering J. Invest Dermatol. 127:2098-2100.
Chuong, C. M., McClain, D. A., Streit, P., and Edelman, G. M. (1982) Neural cell adhesion molecules in rodent brains isolated by monoclonal antibodies with cross-species reactivity. Proc Natl Acad. Sci. 79, 4234.
Gurdon, J. B., and Melton, D. A. (2008) Nuclear reprogramming in cells. Science 322, 1811.
Havlickova B, Biro T, Mescalchin A, Tschirschmann M, Mollenkopf H, Bettermann A, et al. (2009) A human folliculoid microsphere assay for exploring epithelial-mesenchymal interactions in the human hair follicle. J Invest Dermatol 129:972-983.
Jiang, T.-X., Jung, H. S., Widelitz, R. B., Chuong, C.-M. (1999) Self organization of periodic patterns by dissociated feather mesenchymal cells and the regulation of size, number and spacing of primordia. Development 126:4997-5009.
Kremer M., Lang, E., Berger, A C (2000) Evaluation of dermal-epidermal skin equivalents (‘composite skin’) of human keratinocytes in a collagen-glycosaminoglycan matrix (Integra Artificial Skin). British Journal of Plastic Surgery 53.459-465.
Lee L F, Chuong C M (2009) Building complex tissues: high-throughput screening for molecules required in hair engineering. The Journal of investigative dermatology 129:815-817.
Lichti U, Weinberg W C, Goodman L, Ledbetter S, Dooley T, Morgan D, et al. (1993) In vivo regulation of murine hair growth: insights from grafting defined cell populations onto nude mice. J Invest Dermatol 101:124 S-129S.
Lichti, U., Anders, J., and Yuspa, S. H. (2008) Isolation and short-term culture of primary keratinocytes, hair follicle populations and dermal cells from newborn mice and keratinocytes from adult mice for in vitro analysis and for grafting to immunodeficient mice. Nature protocols 3, 799.
Ma, L., Liu, J., Wu, T., Plikus, M., Jiang, T.-X, Liu, Y.-H., Muller-Rover, S., Peters, H., Maxson, R., Maas, R., and Chuong, C-M. (2003) Cyclic alopecia in Msx2 mutants: Defects in hair cycling and hair shaft differentiation. Development 130:379-389.
Maini, P K, Baker, R E, Chuong, C M. (2006) The Turing model comes of molecular age. Science. 314: 1397-1398.
Morris R J, Liu Y, Manes L, Yang Z, Trempus C, Li S, Lin J S, Sawicki J A, Cotsarelis G. (2004) Nat. Biotechnol. 22(4):411-7.
Nakao K, Morita R, Saji Y, Ishida K, Tomita Y, Ogawa M, et al. (2007) The development of a bioengineered organ germ method. Nature methods 4:227-230.
Plikus, M. V., and Chuong, C. M. (2008) Complex hair cycle domain patterns and regenerative hair waves in living rodents. J Invest Dermatol. 128, 1071.
Plikus, M. V., Mayer, J. A., de la Cruz D, Baker, R. E., Maini, P. K., Maxson, R., and Chuong, C. M. (2008) Cyclic dermal BMP signalling regulates stem cell activation during air regeneration. Nature 451, 340.
Powell, H. M., and Boyce, S. T. (2009) Engineered human skin fabricated using electrospun collagen-PCL blends: morphogenesis and mechanical properties. Tissue Eng Part, 15, 2177.
Qiao J, Turetsky A, Kemp P, Teumer J (2008) Hair morphogenesis in vitro: formation of hair structures suitable for implantation. Regenerative medicine 3:683-692.
Stenn K S, Cotsarelis G (2005) Bioengineering the hair follicle: fringe benefits of stem cell technology. Curr Opin Biotechnol 16:493-497.
Tumbar T, Guasch G, Greco V, Blanpain C, Lowry W E, Rendl M, Fuchs E. (2004) Defining the epithelial stem cell niche in skin, Science 303(5656):359-63.
Yen C M, Chan C C, Lin S J. (2010) High-throughput reconstitution of epithelial-mesenchymal interaction in folliculoid microtissues by biomaterial-facilitated self-assembly of dissociated heterotypic adult cells. Biomaterials 31(15):4341-52.
Yeh J, Green L M, Jiang T X, Plikus M, Huang E, Chang R N, Hughes M W, Chuong C M, Tuan T L (2009) Accelerated Closure of Skin Wounds in Mice Deficient in the Homeobox Gene Msx2. Wound Repair and Regeneration. 17:639-648.
Young T H, Lee C Y, Chiu H C, Hsu C J, Lin S J (2008) Self-assembly of dermal papilla cells into inductive spheroidal microtissues on poly(ethylene-co-vinyl alcohol) membranes for hair follicle regeneration. Biomaterials 29:3521-3530.
Young T H, Tu H R, Chan C C, Huang Y C, Yen M H, Cheng N C, et al. (2009) The enhancement of dermal papilla cell aggregation by extracellular matrix proteins through effects on cell-substratum adhesivity and cell motility. Biomaterials 30:5031-5040.
Zheng Y, Du X, Wang W, Boucher M, Parimoo S, Stenn K (2005) Organogenesis from dissociated cells: generation of mature cycling hair follicles from skin-derived cells. J Invest Dermatol 124:867-876.

What is claimed is:

1. A method for preparing pilosebaceous units in a physiological plane, comprising admixing a number of skin precursor cells and a suitable medium, wherein the concentration of skin precursor cells present in the medium is from about 0.5 million cells per 100 μl medium to about 40 million cells in 300 μl medium.

2. The method of claim 1, wherein the concentration of cells in the medium is from about 2 million cells per 150 μl medium to about 20 million cells in 200 μl medium.

3. The method of claim 1 or 2, further comprising culturing the cells in the medium for an effective amount of time to allow the cells to settle and excess liquid to be removed.

4. The method of claim 1, wherein the effective amount of time is from about 30 minutes to about 4 hours at a temperature that supports cell stability.

5. The method of claim 4, wherein the temperature in a range from about 34° C. to about 40° C.

6. The method of claim 4, wherein the temperature in a range from about 36° C. to about 38° C.

7. The method of claim 1, wherein the skin precursor cells comprise epidermal and dermal precursor cells.

8. The method claim 1, wherein the epidermal and dermal precursor cells are isolated or purified cells from neonatal or aged mammals.

9. The method of claim 7 or 8, wherein the ratio of epidermal to dermal precursor cells is from about 1:3 to about 1:15.

10. The method of claim 7 or 8, wherein the ratio of epidermal to dermal precursor cells is from about 1:5 to about 1:10.

11. The method of claim 1, further comprising admixing an effective amount of an agent inhibiting Bone Morphogenic Protein (BMP) signaling.

12. The method of claim 11, wherein the agent is selected from the group consisting of dorsomorphin, noggin, chordin, gremlin, sclerostin and follistatin and combinations thereof.

13. The method of claim 1, further comprising admixing an effective amount of an agent promoting cell differentiation or growth.

14. The method of claim 13, wherein the agent is selected from the group consisting of Platelet Derived Growth Factor (PDGF), Vascular Endothelial Growth Factor (VEGF), Epithelial Growth Factor (EGF), TGF-, Fibroblast Growth Factor (FGF), insulin, transferrin, retinoid and combinations thereof.

15. The method of claim 1, further comprising an effective amount of minoxidil, finasteride, or an agent enhancing cell growth.

16. The method claim 3, further comprising seeding the cells in the medium onto a biocompatible scaffold.

17. The method of claim 16, wherein the biocompatible scaffold is dried or lyophilized prior to admixing with the cells in serum-free medium.

18. The method of claim 16, wherein the cells are seeded by passively contacting the cells with the scaffold at a temperature range from about 25° C. to about 40° C. for about 30 minutes to about 2 hours.

19. The method of claim 3, further comprising implanted into the cells in the suitable medium onto or into the dermis of the non-human animal.

20. The method of claim 1, further comprising admixing an agent to be screened, and monitoring the growth of hair in vitro or in vivo.

21. A method of claim 1, further comprising implanting the cells and suitable medium into the dermal layer of the mammal under a condition that favors implantation of the composition into the dermis of the mammal.

22. The method of claim 21, wherein the condition that favors implantation of the composition into the dermis of the mammal comprises applying suitable pressure to maintain contact between the composition and the muscle or subcutaneous fat of the mammal for at least 3 days.

23. The method of claim 21 or 22, wherein the dermal layer of the mammal was pretreated with an effective amount of an agent inhibiting the Bone Morphogenic Protein (BMP) signaling.

24. The method of claim 23, wherein the agent is one or more of dorsomorphin, noggin, chordin, gremlin, sclerostin or follistatin.
